(19) United States
(12) Patent Application Publication
(10) Pub. No.: US 2012/0046356 A1
(43) Pub. Date: Feb. 23, 2012

(54) NEW PROCESS FOR PREPARING DIKETONES AND MEDICAMENTS

(75) Inventor: Lars Eklund, Karlskoga (SE)

(73) Assignee: CAMBREX KARLSKOGA AB, Karlskoga (SE)

(21) Appl. No.: 13/122,208

(22) PCT Filed: Oct. 2, 2009
(86) PCT No.: PCT/GB2009/002346

§ 371 Date: Apr. 1, 2011

Related U.S. Application Data
(60) Provisional application No. 61/202,812, filed on Apr. 8, 2009.

(30) Foreign Application Priority Data
Oct. 2, 2008 (GB) PCT/GB2008/003341

(51) Int. Cl. A61K 31/343; A61P 9/06; C07C 49/825; C07D 307/80; C07C 45/00; C07C 45/81

(52) U.S. Cl. 514/469; 568/314; 568/324; 568/337; 549/468; 428/402

(57) ABSTRACT

There is provided a process for the preparation of a compound of formula (III), wherein X and Y are as described in the description. Such compounds may, for example, be useful intermediates in the synthesis of drugs such as Dronedarone. There is also provided a process for the preparation of a compound of formula (I).

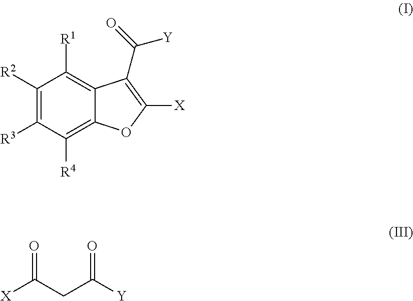

The present invention relates to a process for the manufacture of a certain diketone, which is a useful intermediate in synthesis of compounds, especially drugs, such as anti-arrhythmia drugs, e.g. Dronedarone (N-({2-(n-butyl)-3[4-(3-dibutylamino-propoxy)-benzoyl]-benzofuran-5-yl}methane-sulfonamide).

Dronedarone is a Class III anti-arrhythmia drug for the prevention of cardiac arrhythmias such as atrial fibrillation (AF). AF is a condition characterised by an irregular heart beat and occurs when the atria (the upper chambers of the heart) contract very rapidly. This causes the lower chambers of the heart, the ventricles, to contract chaotically so that blood is inefficiently pumped to the body which can lead to tissue damage and even death.

Dronedarone is prepared via a stepwise procedure which involves the synthesis of a number of intermediates, including 2-butyl-3-(4-methoxybenzoyl)-5-nitrobenzofuran and 2-butyl-3-(4-hydroxybenzoyl)-5-nitrobenzofuran.

2-Butyl-3-aroyl-5-nitrobenzofurans are typically synthesised via Friedel-Craft acylation of 3-unsubstituted 2-butyl-5-nitrobenzofurans. Such reactions are described in U.S. Pat. No. 5,223,510 and U.S. Pat. No. 5,854,282, Japanese patent document JP 2002-371076 and international patent application WO 2007/140989. Given that there is no disclosure in these documents of a benzofuran forming reaction that results directly in a 3-acyl-benzofuran, there is also no disclosure of any diketone intermediate that may be employed in such a reaction.

Diketones have been synthesised from 4-hydroxy-acetophenone, in which the hydroxy group is first acylated (typically by reaction with an acid anhydride), and then an intramolecular condensation reaction occurs, in the presence of an additive such as BF₃. Such reactions are described in e.g. EP 900 831. UK patent application GB 948 494 also describes a reaction of a phenolic ketone with an acid anhydride. Such reactions are performed in the presence of an alkali metal, such as sodium, or BF₃, with the resulting diketone being isolated as a complex. Journal article by El-Ansary, A. K., Egyptian journal of pharmaceutical sciences (1991), 32 (3-4), page 709-17, Jones et al, Journal of Chem. Soc., Perkin Trans 2 (1975), 11, p 1231-4 and Jones et al., Makromolekulare Chemie (1961)m 50, p 232-43 all disclose the synthesis of various diketones. However, there is no specific disclosure of a condensation reaction to form a diketone, in which one of the reactants is a hydroxyphenyl group, in which the hydroxy group is unprotected.

Various documents also described the purification of a diketone, such as U.S. patent application Ser. No. 10/470,893, which involves the formation of a complex. There are no disclosures of a technique that involves crystallisation.

International application WO 2009/044143 discloses the synthesis of 1-(4-hydroxyphenyl) heptane-1,3-dione, which is prepared via a protected hydroxyphenyl compound, and has an aggregate particle size of about 230×120 μM.

There is a need for syntheses of diketones that are more efficient or otherwise advantageous over known syntheses. Such diketones may be used to prepare 3-aroylbenzofurans directly, i.e. by-passing the formation of a 3-unsubstituted benzofuran, and therefore circumventing the need for a Friedel-Crafts acylation step.

The listing or discussion of an apparently prior-published document in this specification should not necessarily be taken as an acknowledgement that the document is part of the state of the art or common general knowledge.

In a first aspect of the invention, there is provided a process for the preparation of a compound of formula III,

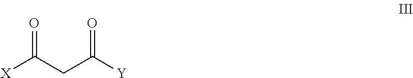

or a derivative thereof, wherein:
X represents hydrogen or C₁₋₆ alkyl optionally substituted by one or more halo (e.g. fluoro) atoms;
Y represents aryl or heteroaryl substituted by at least one (e.g. one) —OH group;
   which process comprises reaction of a compound of formula VII,

Y—C(O)—CH₃ VII

or a derivative thereof, wherein Y is as defined above, characterised in that the requisite —OH substituent thereon is not protected, with a compound of formula VIII,

X—B¹ VIII

or a derivative thereof, wherein:
X is as defined above;
B¹ represents —C≡N or, preferably, —C(O)L¹;
L¹ is a suitable leaving group, such as halo (e.g. bromo, chloro or iodo) or, more preferably, —OC₁₋₆ alkyl (e.g. —OCH₃ or, preferably, —OCH₂CH₃),
in the presence of base, wherein the base comprises an alkali metal alkoxide, in which the alkyl moiety of the alkoxide is a branched C₃₋₆ alkyl group, or the like (i.e. equivalents of such a base),
   which process is hereinafter referred to as “the process of the invention”.

As stated hereinbefore, the reaction is characterised in that in the compound of formula VII, the requisite —OH substituent on the aryl or heteroaryl group defined by the integer Y is not protected. By this we mean that that group exists as a free —OH group or, in another embodiment, as a salt thereof, such as a moiety of formula —O⁻A⁺ in which A represents a Group I alkali metal, e.g. potassium or, preferably sodium, so forming e.g. a —O⁻Na⁺ moiety (however, the —OH group is not covalently bonded to another atom, such as a carbon atom). Preferably therefore, in the compound of formula III that is produced by the process of the invention, the corresponding —OH is also not protected (but may exist as —O⁻A⁺ or in the free —OH form; in practice, the reaction of the process of the invention will be quenched with a proton and hence any compound of formula III formed in situ in which there is a —O⁻A⁺ present may be converted to, and isolated as, a corresponding compound of formula III in which there is a free —OH group present).

The process of the invention may be performed employing salts, solvates or protected derivatives (e.g. in which the carbonyl group is protected, as an imine) of the compounds of formulae VII and VIII. Compounds of formula III that may thereby be produced may or may not be produced in the form of a (e.g. corresponding) salt or solvate, or a protected derivative thereof (for example a protected carbonyl group, such as an imine may be produced). However, as stated hereinbefore, the requisite —OH substituent attached to the aryl or heteroaryl group in the Y group of the compound of formula VII may not be ‘derivatised’, i.e. it may not be protected (e.g. by being covalently bonded via a carbon atom), but exists as the free —OH group (or salt thereof). The skilled person will appreciate that when a compound of formula VIII in which B¹ represents —C≡N is employed, then the resultant product of formula III so formed by the process of the invention may necessarily be one in which a carbonyl group is protected as an imine (e.g. a compound of formula III that is X—C(═NH)—CH₂—C(═O)—Y, or a derivative, or the like may be formed), in which the imino (═NH) moiety may be hydrolysed to give a compound of formula III that is X—C(═O)—CH₂—C(═O)—Y. Most preferably, a compound of formula VIII in which B¹ represents —C(O)L¹ is employed in the process of the invention.

Compounds employed in or produced by the processes described herein (i.e. those involving the process of the invention) may exhibit tautomerism. The process of the invention therefore encompasses the use or production of such compounds in any of their tautomeric forms, or in mixtures of any such forms. For example, the compound of formula III may exhibit keto-enol tautomerism.

Similarly, the compounds employed in or produced by the processes described herein (i.e. those involving the process of the invention) may also contain one or more asymmetric carbon atoms and may therefore exist as enantiomers or diastereoisomers, and may exhibit optical activity. The process of the invention thus encompasses the use or production of such compounds in any of their optical or diastereoisomeric forms, or in mixtures of any such forms.

Further, the compounds employed in or produced by the processes described herein (e.g. compounds of formula IIA) may contain double bonds and may thus exist as E (entgegen) and Z (zusammen) geometric isomers about each individual double bond. All such isomers and mixtures thereof are included within the scope of the invention.

Unless otherwise specified, alkyl groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms, such alkyl groups may also be part cyclic/acyclic. Such alkyl groups may also be saturated or, when there is a sufficient number (i.e. a minimum of two) of carbon atoms, be unsaturated.

The term “aryl”, when used herein, includes C₆₋₁₄ (e.g. C₆₋₁₀) groups. Such groups may be monocyclic, bicyclic or tricyclic and, when polycyclic, be either wholly or partly aromatic. C₆₋₁₀ aryl groups that may be mentioned include phenyl, naphthyl, and the like. For the avoidance of doubt, the point of attachment of substituents on aryl groups may be via any carbon atom of the ring system.

The term “heteroaryl”, when used herein, includes 5- to 14-membered heteroaryl groups containing one or more heteroatoms selected from oxygen, nitrogen and/or sulfur. Such heteroaryl group may comprise one, two or three rings, of which at least one is aromatic. Substituents on heteroaryl groups may, where appropriate, be located on any atom in the ring system including a heteroatom. The point of attachment of heteroaryl groups may be via any atom in the ring system including (where appropriate) a heteroatom. Examples of heteroaryl groups that may be mentioned include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, quinolinyl, benzoimidazolyl and benzthiazolyl.

The term “halo”, when used herein, includes fluoro, chloro, bromo and iodo.

In the process of the invention, preferred compounds of formula III that may be produced include those in which:
X represents C₁₋₄ alkyl (optionally substituted by one or more fluoro atoms; but preferably, unsubstituted), for example C₄ alkyl, such as 1-methylpropyl, or, most preferably, butyl (especially n-butyl);
Y represents phenyl substituted by one —OH group (or a salt thereof, e.g. a —O⁻Na⁺ group) in the 2-, 3- or, preferably, in the 4-position;
L¹ preferably represents a suitable leaving group such as halo (e.g. bromo, chloro or iodo) or, more preferably, —OC₁₋₆ alkyl (e.g. —OCH₃ or, preferably, —OCH₂CH₃);
   however, equivalent leaving groups may be employed.

Most preferred compounds of formula III include those in which:
Y represents 4-(OH)-phenyl (Y may also be substituted by other substituents in addition to the requisite —OH group, for instance other small substituents such as halo, —OH, —O—C₁₋₃ alkyl (which alkyl group is optionally substituted by one or more fluoro atoms) and/or —CN, but Y is preferably not substituted by other substituents);
X represents n-butyl;
B¹ represents —C(O)OCH₂CH₃.

The process of the invention is performed in the presence of a certain alkali metal alkoxide. Preferably, the alkali metal is a Group I metal, such as potassium or, preferably sodium. It is stated that the alkoxy moiety of the base is branched. Preferably, the branching occurs at the position a to the carbon atom that is attached to the requisite oxygen atom of the alkoxy group (and hence, the C₃₋₆ alkyl group is secondary or, preferably, tertiary, relative to the point of attachment to the oxygen atom). Most preferably, the alkoxy moiety is branched C₄₋₆ alkyl (e.g. tert-butyl). The most preferred base is sodium tert-butoxide. Such bases in which the alkyl moiety of the alkali metal alkoxide is branched possess a higher pKa (i.e. are stronger bases) than corresponding bases in which the alkyl moiety is not branched, but linear (corresponding bases containing a primary alkyl group, relative to the point of attachment to the oxygen atom).

The base employed in the process of the invention is one that possesses a certain pKa. Similarly, other suitable bases that possess a similar, or higher, pKa may also be employed in the process of the invention (which bases are referred to herein as equivalent bases to the requisite alkali metal alkoxide base employed in the process of the invention). Such bases are advantageous in the process of the invention, as they may improve the yield and efficiency of the process, for example by reducing side reactions and therefore undesired by-products (e.g. reducing competing condensation reactions, e.g. self-condensations). When the compound of formula VII contains a free —OH group, this (i.e. the reduction of side reactions) may be due to accompanying deprotonation of that hydroxy group, which forms an alkali metal salt (i.e. —O⁻A⁺), Which may make it less reactive to carbonyl groups, thereby decreasing the likelihood of self-condensation.

As stated hereinbefore, a certain alkali metal alkoxide is employed in the process or another suitable base (e.g. equivalent base). By another suitable base, we mean that that base possesses a similar, or higher, pKa to the alkali metal alkoxide employed in the process of the invention, or, exerts a similar effect to it, for example by promoting the reaction by a similar mechanism. Other suitable bases that may be employed include any of the following: another alkali metal based base (e.g. a carbonate base, such as Na₂CO₃ or K₂CO₃ and/or a phosphate base, such as K₃PO₄), an alkali metal hydride (e.g. KH, CaH₂ or, preferably, NaH), an organolithium base (e.g. n-, s- or t-butyllithium or, preferably, lithium diisopropylamide), or mixtures of bases.

The process of the invention requires the presence of a certain alkali metal alkoxide (or the like), although other bases may also be present in the reaction mixture. Preferably, however, the process is performed predominantly in the presence of the requisite alkali metal alkoxide base (or equivalent thereof) as the base in the reaction mixture (e.g. in the number of equivalents as defined herein), and, optionally (e.g. in the case where there is a free —OH group present on the Y group in the compound of formula VII), in the presence of a base, e.g. at least, or about, one equivalent that is able to deprotonate that —OH moiety (for example as defined herein).

For example when the compound of formula VII contains a free —OH group, it is preferred that at least, or about, one equivalent of base (e.g. the requisite alkali metal alkoxide, or the like) is employed (equivalent to the molar quantity of the compound of formula VII). However, as the first equivalent of base may deprotonate the free —OH group of the compound of formula VII (thereby forming a corresponding compound of formula VII in which there is a —O⁻A⁺ moiety present), then it is preferred that at least 1.5 and preferably at least, or about, 2 equivalents of base are employed, if yield is to be maximised. Most preferably, however, at least 2.5, e.g. at least, or about, 3 equivalents of base (e.g. the requisite alkali metal alkoxide, or the like) is employed, in order to maximise yield, as the compound of formula III to be formed may enolise, and therefore may require an additional one equivalent of base. Preferably, all of the base employed in the process of the reaction is the requisite alkali metal alkoxide, or equivalent thereof, as defined herein. However, mixtures of different bases may be employed, provided that at least, or about, one equivalent, e.g. at least, or about, 2 (and preferably at least, or about, 3) equivalents of the requisite alkali metal alkoxide (or equivalent) is employed.

When the compound of formula VII contains a —O⁻A⁺ moiety (instead of the free —OH group, in which A⁺ is a group I metal anion, preferably, Na⁺) then one less equivalent of base may be required (as the free —OH moiety has already been deprotonated), and hence, the amount of base (e.g. the requisite alkali metal alkoxide, or equivalent) is preferably, at least, or about, one equivalent, and preferably, at least, or about, 2 equivalent. As stated hereinbefore, the compound of formula VII in which there is a —O⁻A⁺ moiety present may be prepared in situ by reaction with the requisite alkali metal alkoxide base present in the process of the reaction. However, such a compound may be pre-formed, or may be formed in situ by reaction with another suitable alkali metal base first (followed by the reaction with the compound of formula VIII and requisite alkali metal alkoxide base, or equivalent), in which case suitable bases include alkali metals (such as sodium, e.g. sodium wire) or strong alkali metal bases such as alkali metal hydroxides (e.g. potassium or, preferably, sodium hydroxide; in which latter case a —O⁻Na⁺ moiety is formed).

The process of the invention may be performed in the presence of (a) suitable solvent(s) (such as tetrahydrofuran (THF), toluene and/or dimethylformamide; a polar aprotic solvent such as THF is particularly preferred). However, in the case where one of the reactants (e.g. compound of formula VIII) is a liquid at the reaction temperature, then the reaction may also be performed in the absence of solvent (as the reactant, e.g. compound of formula VIII, may serve as solvent).

As stated hereinbefore, the product (of compound III) formed by the process of the invention may be in the form of an enolate. Hence, the reaction of the process of the invention is preferably quenched by the addition of an appropriate quantity (e.g. at least one equivalent) of a proton source, e.g. a protic acid, such as a hydrogen halide (e.g. HCl) or a weak organic acid (e.g. a carboxylic acid, such as acetic acid). Advantageously, when a weak organic acid is employed, the quench may also result in crystallisation/precipitation of the product, for example, as defined hereinafter.

The process of the invention may be performed in the presence of any quantity of each of the compounds of formulae VII and VIII. However, it is preferably performed in the presence of compounds of formulae VII and VIII that are in a molar ratio of from about 3:2 to about 2:3, and most preferably in a molar ratio of from about 1.1:1 to about 1:1.1 (e.g. about 1:1).

The process of the invention may be performed under standard reaction conditions, such as at room temperature or elevated temperature (e.g. about 40° C.), such as about 65° C., or above (e.g. between about 40° C. and 85° C.). Other specific temperatures that may be mentioned are between about 68° C. and 73° C. (e.g. at about 70 to 73° C.). The length of the reaction may be determined by the skilled person (e.g. by monitoring the extent of reaction by tic). However, preferably, the reaction of the compound of formula VII with the compound of formula VIII (to form a compound of formula III) may take more than 2 hours, for instance at least, or about, 6 hours, and even at least, or about, 15 hours.

In the process of the invention, compound of formula VII and VIII may be mixed together. This mixture may be added to the base employed in the process of the invention (which base is optionally, and preferably, present in solvent that may be employed in the process of the invention) or vice versa, i.e. the base (and solvent) is added to the mixture of compound of formula VII and VIII.

As stated hereinbefore, the process of the invention may be quenched by the addition of a proton source (e.g. a carboxylic acid, such as acetic acid). The proton source (e.g. a carboxylic acid, such as acetic acid) may be present as a mixture with water. In such instances (which are preferred), the amount of the carboxylic acid is such that there is at least one mole of carboxylic acid (e.g. acetic acid) per mole of compound of formula VII or formula VIII (more preferably, there is present at least two molar equivalents of the carboxylic acid, e.g. at least, or about, three molar equivalents of carboxylic acid (e.g. acetic acid)). The amount of water that may be (and preferably is) mixed with the carboxylic acid is preferably at least, or about, 100 g water per mole of compound of formula VII or VIII (for instance, at least, or about, 200 g (e.g. 300 or preferably 400 g, e.g. 450 g) per mole of compound of formula VII or formula VIII, or, the amount of water is at least, or about, 50 g per mole of carboxylic acid that may be present as the proton source (for instance at least or about 100 g, e.g. about 150 g, per mole of carboxylic acid). As stated hereinbefore, the carboxylic acid may be mixed with water. It is particularly advantageous therefore that the carboxylic acid and water are miscible.

Advantageously, and preferably after the reaction of the process of the invention is quenched as described above, any solvent (e.g. THF; and other volatile substance present in the reaction mixture, such as the proton source, for instance if it is a volatile carboxylic acid such as acetic acid), may be recovered (and optionally re-used) by concentration in vacuo or, preferably, at atmospheric pressure in which case the mixture is heated e.g. to above the reaction temperature, e.g. to above about 80° C. (e.g. above, or about, 90° C., preferably at about 100° C., e.g. 102° C.). This is particularly important from an economical and/or environmental point of view. Thereafter, the temperature of the reaction mixture may be cooled (e.g. to about 75° C.) and the water phase may be separated. Then (i.e. after any volatiles that may be re-used are already separated), the reaction mixture/reaction vessel may be heated again under vacuum in order to remove any other undesired products, e.g. unreacted starting material, such as compound of formula VIII. This procedure to remove other undesired product may be stopped or interrupted when the liquid temperature is at least, or about, 110° C. at a pressure of at most, or about, 50 mbar.

After the reaction of the process of the invention is quenched (e.g. as described above), and volatiles stripped off, the desired compound of formula III may be isolated by a standard work-up procedure (e.g. by extraction with a suitable organic solvent, such as toluene). However, advantageously, it has been found that yield of the compound of formula III obtained may be increased/maximised by following certain procedures, for instance by the addition of further water and proton source (e.g. carboxylic acid such as acetic acid) for instance after volatiles/other undesired products (e.g. that may have been removed by heating at atmospheric pressure or under vacuum). For instance, at least one mole of carboxylic acid (e.g. acetic acid) per mole of compound of formula VII or formula VIII (more preferably, there is present at least two molar equivalents of the carboxylic acid, e.g. at least, or about, three or four molar equivalents of carboxylic acid (e.g. acetic acid)) may be added, and the amount of water may be at least, or about 25 g per mole of compound of formula VII or VIII (for instance, at least, or about, 75 g (e.g. at least, or about, 100 g) per mole of compound of formula VII or formula VIII. Such a mixture may then be cooled, for instance to about room temperature (e.g. between about 25 and 28° C.). The mixture may advantageously be further cooled to below room, temperature, e.g. below 0° C., such as below, or about −10° C., e.g. about −12° C. Further water is preferably added at this low temperature (e.g. at least, or about 25 g (e.g. at least, or about, 50 to 75 g) per mole of compound of formula VII or VIII). Advantageously, this work-up procedure may result in a higher yield of the desired product of formula III, which may be isolated by standard methods, e.g. simply by filtration. Thereafter the filter cake may be washed (e.g. with diluted carboxylic acid, 20% acetic acid, and subsequently with water), and dried (e.g. under vacuum, optionally at elevated temperature, e.g. at about 50° C.).

Surprisingly, the process of the invention proceeds without the need to protect and deprotect the hydroxy group. The process of the invention may therefore be more efficient and/or economical. It may also thereby provide environmental advantages. Advantageously, the unprotected hydroxy group does not substantially interfere with the process of the reaction, which may normally be considered to be likely given that the hydroxy moiety (of the compound of formula VII) may act as a nucleophile, which may attract reaction with the carbonyl group of another separate molecule of the compound of formula VII, thereby producing an undesirable side-reaction. Surprisingly, in the process of the reaction, the ketone of formula VII is less prone to undesirable side-reactions (e.g. self-condensation reactions).

Preferably, the reaction is performed in the absence of a further additive such as a boron reagent (such as BF₃ or BF₂, or a complex thereof). Further, the compound of formula III produced by the process of the invention is not isolated as a complex, for example (a) copper chelate(s).

In a further aspect of the invention, there is provided a process for the isolation/purification of a compound of formula III, as hereinbefore defined, which process comprises crystallisation or precipitation of the compound, in a solvent system, which is hereinafter also referred to as a process of the invention.

Crystallisation (or precipitation) of the compounds prepared by the process of the invention may be performed in any suitable solvent (or mixtures of solvents). However, it has preferably surprisingly been found that certain solvent systems are particularly preferred. Particularly preferred solvent systems for the crystallisation or precipitation of the compound of formula III include an aqueous solvent and weak organic acids (such as a carboxylic acid as defined herein, e.g. formic, propionic, or preferably, acetic acid).

The crystallisation/precipitation process of the invention described herein has the additional advantage that the compound of formula III may be present in the reaction mixture with other products (e.g. unreacted starting material or other undesired side-products), but this purification/isolation process may still proceed. For example, the compound of formula III may be present in less than 95% (e.g. less than 90%, such as less than, or about, 80%) of the mixture to be crystallised/precipitated, but the isolated/purified product so formed may not contain those undesired products (and may be present in a higher percentage, such as above 95%, e.g. above 99%, such as near, or at, 100%, in the product formed).

Most preferably, the solvent system employed in the crystallisation or precipitation process comprises a mixture of water and a weak organic acid (e.g. a carboxylic acid such as acetic acid). When such a mixture of solvents is employed in the solvent system, then any ratios may be employed, for instance between 1:10 and 10:1 of water:weak organic acid. However, preferably, the ratio is between 1:5 and 5:1, for example between 1:3 and 3:1 and, especially, about 1:1.

Preferably, the crystallisation solvent is homogenous, for example the solvents may forms an azeotropic mixture. However, a suitable solvent may also be employed as an “anti-solvent” (i.e. a solvent in which salts of compounds of formula I are poorly soluble) in order to aid the crystallisation process.

Crystallisation temperatures and crystallisation times depend upon the concentration of the compound in solution, and upon the solvent system which is used.

Surprisingly, it has been found that the crystallisation or precipitation of the process of the invention produces a new physical form of a compound of formula III. Hence, in a further aspect of the invention, there is provided a compound of formula III obtainable by the crystallisation/precipitation of the process of the invention described herein.

In a further aspect of the invention, there is provided a compound of formula III as hereinbefore defined (e.g. one that is not a derivative of formula III), wherein the average particle size is at least 250×150 μM (also referred to herein as an aspect of the invention, and a process for preparing such a product is also referred to herein as another process of the invention). Preferably, the average particle size is at least 300×200 μM (e.g. at least 400×300 μM, for example about 500×380 μM). Such compounds may be inherently larger than those described in the prior art. “Average” when referred to herein refers to the median. The measurements may be taken on particles that are (or are close to) rectangular (and hence the larger figure refers to the length, and the smaller figure refers to the width). The measurements may also be taken on particles that are (or are close to) spherical, in which case the figures refer to diameters (or cross-section). The measurements are preferably taken on ‘individual’ particles, rather than ‘clustered’ particles. These measurements assume that a large proportion (e.g. the majority) of the (‘individual’) particles are substantially rectangular, spherical, oval or oblong in shape (this is preferably the case, for instance when such particles are prepared by the process(es) of the invention described hereinbefore). This may be shown by the figures hereinafter, which compare the particles produced by the processes of the invention described herein, with the particles produced by the process step of Example A (Example 1, (b); where the diketone of formula III is produced by benzyl deprotection of the hydroxy moiety) of international patent application WO 2009/044143.

The new physical form (with increased average particle size) may lead to advantages in terms of handling of the compound of formula III and/or improvements in the characteristics of the compound.

The formation of a particular crystalline salt of a compound may be advantageous (as compared to, for example, an amorphous form), as crystalline forms may be easier to purify and/or handle. Crystalline forms may also have a better solid state stability and shelf-life (e.g. be stored for longer periods of time without substantial change to the physico-chemical characteristics, e.g. chemical composition, density and solubility).

The skilled person will appreciate that, if a compound can be obtained in stable crystalline form, then several of the above-mentioned disadvantages/problems with amorphous forms may be overcome. It should be noted that obtaining crystalline forms is not always achievable, or not easily achievable. Indeed, it is typically not possible to predict (e.g. from the molecular structure of a compound), what the crystallisation behaviour of a certain compound, or a salt of it, may be. This is typically only determined empirically.

In a further embodiment of the invention, there is provided a combination of the processes of the invention described herein. For example, there is provided a process for the preparation of a compound of formula III (which comprises reaction of a compound of formula VII and VIII, as hereinbefore defined; referred to hereinafter as process (i)) followed by crystallisation (or precipitation) as hereinbefore described (referred to hereinafter as process (ii)). Preferably, process (ii) is performed directly after process (i), for example, by separation of the compound of formula III (e.g. by extraction and removal/evaporation of solvent), following by mixing/contacting the compound of formula III with the solvent system of the crystallisation process. Alternatively, in a further embodiment of the invention, process (ii) can be performed directly after process (i) and in the same reaction pot, e.g. by quenching process (i) in the solvent system required for process (ii).

Advantageously, the compound of formula III, prepared by the process of the invention may be employed to prepare a compound of formula I,

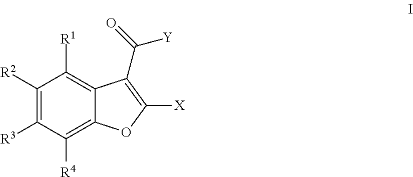

wherein R¹, R², R³ and R⁴ independently represent hydrogen, halo, —NO₂, —CN, —C(O)₂R^{x1}, —OR^{x2}, —SR^{x3}, —S(O)R^{x4}, —S(O)₂R^{x5}, —N(R^{x6})R^{x7}, —N(R^{x8})C(O)R^{x9}, —N(R^{x10})S(O)₂R^{x11} or R^{x12};
X represents hydrogen or C₁₋₆ alkyl optionally substituted by one or more halo (e.g. fluoro) atoms (i.e. is as hereinbefore defined);
Y represents aryl or heteroaryl substituted by at least one (e.g. one) —OH group (i.e. is as hereinbefore defined);
R^{x1}, R^{x2}, R^{x3}, R^{x6}, R^{x7}, R^{x8}, R^{x9} and R^{x10} independently represent hydrogen or C₁₋₆ alkyl optionally substituted by one or more halo (e.g. fluoro) atoms;
_{R}^{x4}, R^{x5}, R^{x11} and R^{x12} independently represent C₁₋₆ alkyl optionally substituted by one or more halo (e.g. fluoro) atoms;
   which process comprises reaction of a compound of formula II,

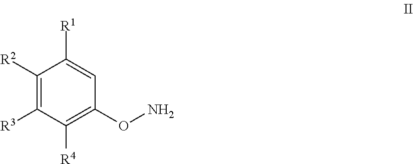

or a protected derivative or salt thereof, wherein R¹, R², R³, R⁴ are as defined above, with a compound of formula III, as prepared by a process of the invention as hereinbefore described,
which process is hereinafter also referred to as “the process of the invention”.

In a further embodiment of the invention, there is provided a process for the preparation of a compound of formula I as hereinbefore defined, but characterised in that:
the reaction is performed as a “one-pot” procedure;
R² represents —NO₂, which process comprises reaction of a compound of formula II prepared by the process of the invention as hereinbefore defined, but in which R² represents —NO₂, with a compound of formula III as hereinbefore defined; or
   the process is performed in the absence of an acylating reagent (for example, when the process of the invention proceeds via an intermediate of formula XXIV (as defined hereinafter), then that intermediate is not first reacted in the presence of an acylating reagent (such as trifluoroacetic anhydride or trifluoroacetyl triflate) to form an N-acylated intermediate in order to promote the pericyclic cyclisation to form the compound of formula I).

For instance, it is specifically stated above that a protected derivative or salt of a compound of formula II may be employed in the process. In this respect, specific salts that may be mentioned include acid salts, such as hydrogen halide salts (e.g. HCl) and specific protecting groups that may be mentioned include suitable protecting groups for the hydroxylamine moiety, such as imino-protecting groups or amino-protecting groups, for example as defined by compounds of formula IIA and IIB,

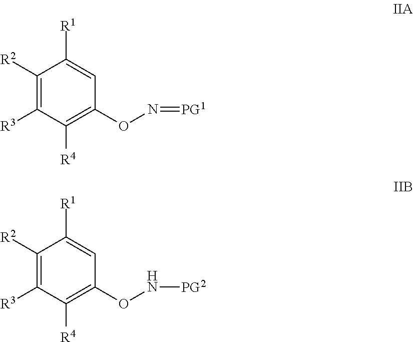

respectively, wherein:
PG¹ represents an imino-protecting group (i.e. a protecting group for the amino moiety that results in an imino functional group), such as ═C(R^{q1})OR^{q2} (so forming a protected hydroxylamine group that is —O—N═C(R^{q1})OR^{q2}), in which R^{q1} and R^{q2} independently represent C₁₋₆ alkyl, and more preferably represent C₁₋₃ alkyl. Most preferably R^{q1} represents methyl and/or R^{q2} represents ethyl (so forming, for example, a compound of formula IIA in which the protected hydroxylamine group is —O—N═C(CH₃)OCH₂CH₃). As stated hereinafter, compounds of formula IIA may exist as geometric isomers, i.e. cis and trans isomers about the imino double bond;
PG² represents an amino protecting group (i.e. a protecting group that results in the amino moiety being a secondary amino group) such as a protecting group that provides an amide (e.g. N-acetyl), N-alkyl (e.g. N-allyl or optionally substituted N-benzyl), N-sutfonyl (e.g. optionally substituted N-benzenesulfonyl) or, more preferably a carbamate or urea.

Hence, PG² may represent:
—C(O)R^{t1} (in which R^{t1} preferably represents C₁₋₆ alkyl or optionally substituted aryl);
C₁₋₆ alkyl, which alkyl group is optionally substituted by one or more substituents selected from optionally substituted aryl;
—S(O)₂R^{t2} (in which R^{t2} preferably represents optionally substituted aryl); or, preferably, —C(O)OR^{t3} (in which R^{t3} preferably represents optionally substituted aryl or, more preferably, C₁₋₆ (e.g. C₁₋₄) alkyl, e.g. tert-butyl (so forming, for example, a tert-butoxycarbonyl protecting group, i.e. when taken together with the amino moiety, a tert-butylcarbamate group);
—C(O)N(R^{t4})R^{t5} (in which, preferably, R^{t4} and R^{t5} independently represent hydrogen, C₁₋₆ alkyl, optionally substituted aryl or —C(O)R^{t6}, and R^{t6} represents C₁₋₆ alkyl or optionally substituted aryl).

When used herein (e.g. in the context of protecting groups such as those defined by PG²), the term “optionally substituted aryl” preferably refers to “optionally substituted phenyl”, in which the optional substituents are preferably selected from halo, —NO₂, —OH and/or —OC₁₋₆ alkyl.

When protected derivates of compounds of formula II are employed in the process of the invention (to produce a benzofuran of formula I), then it is preferred that compounds of formula IIA are employed. However, preferably, compounds of formula IIA are first deprotected, as described herein, to form compounds of formula II, which deprotected compounds are employed in the benzofuran-forming process of the invention.

When protected derivatives of compounds of formula II (e.g. compounds of formula IIA or IIB) or salts of compounds of formula II (e.g. acid salts such as a hydrogen halide salt, e.g. HCl) are employed in the process of the invention, then the step of deprotection to the unprotected compound of formula II, or the step of neutralisation (e.g. by basification of the acid salt) to the free base of the compound of formula II, need not be performed separately (but preferably is), e.g. prior to the process of the invention. Such steps may advantageously be performed in the same “pot” as the process of the invention, i.e. the deprotection or neutralisation may occur whilst the reaction of the process of the invention also occurs, thereby providing compounds of formula I that are not in a protected form and/or not in the form of a salt.

Compounds of formula II, or salts thereof, may be prepared by deprotection of a corresponding compound of formula IIA or IIB, under standard conditions known to those skilled in the art. For instance, for deprotection of compounds of formula IIA, standard hydrolysis conditions may be employed, e.g. the presence of an acid (e.g. a hydrogen halide, such as HBr or, preferably, HCl) in an aqueous solution (the acid may also be an inorganic acid such as phosphorus or sulphuric acid). Such conditions may result in a salt of a (non-protected derivative of a) compound of formula II (e.g. a relevant hydrogen halide salt), or, the free base version of such a compound of formula II (for instance, when the salt form is neutralised, e.g. by basification).

Preferably, when compounds of formula II are prepared from deprotection of compounds of formula IIA (which they preferably are), then such a deprotection step may be performed in the presence of a hydrogen halide, phosphoric acid or sulfuric acid (preferably a hydrogen halide, e.g. HCl) and a solvent system comprising at least 15% by weight of water. Preferably, the solvent system comprises at least 25% by weight of water, for example at least 50% by weight of water. More preferably, the solvent system comprises at least 70% (e.g. at least 80%) and, most preferably, at least 90% by weight water. Most preferably, the solvent system comprises at least 95% water (by weight) and consists essentially of water (for instance, the solvent system consists predominantly, preferably, exclusively of water, e.g. at or near 100% by weight of the solvent system comprises water). Hence, most preferably, the solvent system of the process of the invention consists essentially of water. Provided that it comprises at least 15% water (by weight), the solvent system may also comprise an organic solvent, for example a polar solvent, such as a polar protic solvent, for example an alcohol (e.g. a C₁₋₆ alcohol, such as ethanol or, preferably, methanol), or, more preferably, a polar aprotic solvent such as dioxane, tetrahydrofuran, diethyl ether, dimethoxyethane or, most preferably, acetonitrile. Mixtures of the aforementioned solvents may also be employed. Alternatively, the process of the this aspect of the invention is performed as described herein, but in which the solvent system is one in which water is present in a molar ratio (compared to other solvents in the solvent system) of greater than 1:3, for example, the molar ratio of water:other solvent (in which the other solvent may be an organic solvent, such as an alcohol or, preferably, acetonitrile) is at least 1:2, for example at least 1:1, preferably 2:1. More preferably, the molar ratio of water:other solvent is at least 5:1, e.g. at least 10:1, and most preferably, the molar ratio is greater than 50:1 (for example, the solvent system comprises predominantly, or exclusively, water, as defined herein).

In this aspect of the invention (i.e. preparation of compounds of formula II from compounds of formula IIA), it is preferred that in the process of the invention, the compound of formula IIA is added to the mixture of hydrogen halide, phosphoric acid or sulfuric acid (preferably hydrogen halide, e.g. HCl) and the solvent system employed in the process of the invention. However, in such an embodiment of the invention the whole of the solvent system employed in the process of the reaction need not be mixed with the acid. For example, some of the solvent system may be mixed with the compound of formula IIA (which may aid its addition to the reaction, for example). Further, when organic solvent is present in the reaction mixture, then such solvent may be mixed with the acid, but is preferably mixed with the compound of formula IIA (in order to aid dissolution). However, at least 20% (e.g. at least 30%) of the water present in the solvent system is preferably first mixed with the acid that is employed (e.g. the hydrogen halide; which may exist as hydrogen halide in water as described hereinafter). Preferably, at least 50% (e.g. at least 60%, such as at least 75%) of water that is present in the solvent system is first in admixture with the acid (to which the compound of formula IIA, which may itself be present in solvent, is added). Preferably, in the process of this aspect of the invention, the acid (e.g. hydrogen halide), which may be in the presence of solvent (e.g. water) is mixed/reacted with the compound of formula IIA (which may, optionally be a mixture of compound of formula IIA and the solvent system, as defined herein, e.g. water). As stated above, it is preferred that the compound of formula IIA is added to the acid (e.g. hydrogen halide), optionally in the presence of solvent (e.g. water). Preferably, at least one molar equivalent of hydrogen halide (e.g. HCl) is added in employed, for example, at least, or about, 2 equivalents (preferably at least, or about, 3 equivalents, e.g. at least, or about, 4 equivalents such as about 5 equivalents). It is stated above that the acid, e.g. hydrogen halide (which may be employed as hydrogen halide in an aqueous solution), is reacted/mixed with the compound of formula IIA. Preferably, the compound of formula IIA is added to the acid (e.g. hydrogen halide), both of which may be present in solvent as described herein (e.g. the hydrogen halide is preferably present in an aqueous solution). This addition is preferably performed in portions over a period of time. For example, the compound of formula IIA may be added at such a rate as to maintain the temperature of the reaction (the process of the invention) at a certain level, for example near to room temperature (e.g. or as near as possible to room temperature). Preferably, the temperature of the process of the invention is maintained below about 50° C. (e.g. between about room temperature and 50° C.), such as below about 40° C., e.g. below 35° C. Most preferably, the temperature is maintained at between about room temperature (about 25° C.) and about 32° C. The process of the invention may also be performed at below room temperature, but is preferably performed above 0° C., and is most conveniently performed at about room temperature. The compound of formula IIA may be added to the acid (e.g. hydrogen halide) as a mixture in the solvent system employed in the process of the invention. For example, it may be employed as a mixture of compound of formula IIA in water (for example, as described hereinbefore). The portion-wise addition of the compound of formula IIA to the acid, e.g. hydrogen halide, (or aqueous solution thereof) in the process of the invention is most preferably effected by adding about 1 mole of compound of formula IIA over a period of about 1 hour (e.g. about 0.8 moles over a period of about 50 minutes).

However, the addition need not be portion-wise, i.e. the addition can be substantially as a single “lump-sum”. When the addition is portion-wise, then 1 mole of compound of formula IIA may be added to the acid (e.g. hydrogen halide) over a period of time of between ten minutes and two hours (and is most preferably over a preferred period of about 1 hour, as indicated above). The portion-wise addition may be effected by a continuous addition process over the period of time required, for example, the addition may be via the continuous addition of a compound of formula IIA (in e.g. aqueous solvent) by means of a syringe pump, which may be set to perform the addition at the relevant rate required. The portion-wise addition may also be effected at pre-determined intervals (i.e. non-continuous addition). If the number of moles of compound of formula IIA in the process of the invention is increased or decreased, then the period of time over which the addition occurs may be increased or decreased accordingly (for example, if two moles are employed, then the addition time may be doubled). However, the skilled person will appreciate that other factors may influence the necessary addition period (for example, concentration of the reagents in the solvent and/or temperature; higher concentrations and lower temperatures may reduce the addition period).

The total amount of solvent employed in this aspect of the process of the invention (i.e. to obtain compounds of formula II from deprotection of compounds of formula IIA) should be sufficient for the reaction to proceed (e.g. at a predetermined rate, in order to maximise yield, minimise reaction time, etc). Hence, any suitable amount of solvent may be employed. Preferably, however, the amount of solvent employed in the process of the invention is at least 1%, e.g. at least 10% by weight of the compound of formula IIA (e.g. at least 25%, preferably, at least 50% by weight and especially at least 100% by weight) and/or at least 5% by weight of the acid (e.g. at least 25%, preferably, at least 50% by weight and especially at least 100% by weight) employed in the process of the invention. Alternatively (and particularly when the solvent system comprises predominantly water, e.g. exclusively water), the total amount of solvent present is in an amount that is at least one molar equivalent, compared to the compound of formula IIA. Preferably, there is at least three molar equivalents of solvent present in the solvent system of the process of the invention, e.g. at least five molar equivalents. The actual amount/volume of solvent employed in the process of the invention may be varied, depending on requirements of rate of reaction, yield, etc. There may be any upper limit of the amount of solvent required in the process. However, this may be determined practically so that the reaction mixture is not too dilute (e.g. such that the rate of reaction is too slow) or the quantity is so much that there is excess wastage.

After the deprotection step of the process of this aspect of the invention (i.e. to obtain compounds of formula II from deprotection of compounds of formula IIA) has been effected, then the acidic medium of the reaction mixture may need to be neutralised. As the process of the invention is performed in the presence of acid (e.g. a hydrogen halide, preferably, HCl), then the product of formula of II so formed may exist as an acid (e.g. a hydrogen halide) salt of the compound of formula II. Any acid (e.g. hydrogen halide) salt of the compound of formula II formed by the process of the invention may be neutralised under standard conditions. For example in the presence of a suitable base, for an alkali metal based base, such as an alkali metal hydroxide (preferably sodium hydroxide). For example, the base (e.g. aqueous sodium hydroxide solution), may be between 10 and 50% w/w, e.g. between 15 and 40% w/w, e.g. about 33% w/w). Preferably, the base is added to the mixture of the products of the process of the invention at such a rate at to maintain the temperature of the mixture at a certain level (such as below 50° C.), for example, it is maintained at the same level as the temperature is maintained during the process of the invention, i.e. the temperature is most preferably maintained at between about room temperature (about 25° C.) and about 32° C. Such a neutralisation step, which is encompassed by the scope of the process of the invention, advantageously produces the free-base of the compound of formula II, which may precipitate out of the solvent system (which may comprise the solvent system employed in the process of the invention, e.g. water, and/or any additional solvent employed in the neutralisation step described herein, e.g. water). Hence, the free-base of the compound of formula II so formed may be isolated by standard techniques, e.g. filtration.

Compounds of formula IIB or, preferably, IIA may be prepared by reaction of a compound of formula IV,

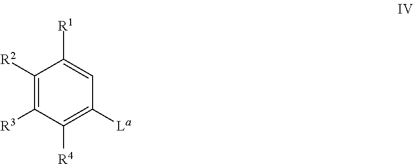

wherein L^{a} represents a suitable leaving group, such as a sulfonate group (e.g. —OS(O)₂CF₃, —OS(O)₂CH₃ or —OS(O)₂PhMe) or, more preferably halo (e.g. bromo, fluoro or, preferably, chloro), and R¹, R², R³ and R⁴ are as hereinbefore defined, with a compound of formula V (in the case of preparation of compounds of formula IIA),

HO—N═PG¹ V

wherein PG¹ is as hereinbefore defined, or a compound of formula VI (in the case of preparation of compounds of formula IIB),

HO—N(H)—PG² VI

wherein PG² is as hereinbefore defined, for example under standard aromatic substitution reaction conditions. For instance, the aromatic substitution reaction may be performed in the presence of a polar aprotic solvent (such as dimethylformamide). In this context, other polar aprotic solvents that may be mentioned include tetrahydrofuran, dimethylsulfoxide, diethyl ether and dioxane. However, it has now been found that this process step may also be performed in a mixture of solvents, only one of which is a polar aprotic solvent (and the other is a non-polar solvent). Hence, in another aspect of the invention, there is provided such a process in the presence of a non-polar solvent, such as a non-polar aprotic solvent, which solvent is employed in addition to the polar aprotic solvent as defined above (and which is preferably dimethylformamide). Preferred non-polar aprotic solvents include toluene, but may be any solvent that may be employed to extract compounds of formula V or VI (e.g. from a reaction mixture as defined hereinafter).

Advantageously, in this aspect of the invention (i.e. the process for the preparation of compounds of formula IIA or IIB), a solution containing the compound of formula V or VI (whichever is employed), for example a solution obtained by the extraction from a reaction mixture (following the preparation of those compounds of formula V or VI), need not be concentrated by the partial or complete evaporation of the solvent (i.e. advantageously, solvent need not be removed). Rather, a polar aprotic solvent (e.g. DMF) may preferably be added directly to a solution of the compound of formula V or VI without complete removal (and most preferably, without any removal) of any non-polar solvent, for example that which is employed in an extraction.

Compounds of formula V in which PG¹ represents ═C(R^{q1})OR^{q2}, may be prepared by reaction of hydroxylamine, or a salt thereof (e.g. a hydrogen halide salt, such as HCl) with a compound of formula XVII,

HN═C(R^{q1})OR^{q2} XVII

wherein R^{q1} and R^{q2} are as hereinbefore defined, under standard reaction conditions. The reaction mixture to obtain such a product may be extracted with a suitable solvent, such as a non-polar solvent (e.g. toluene).

Compounds of formula XVII may be prepared by reaction of a compound of formula XXI,

R^{q1}—CN XXI

wherein R^{q1} is as hereinbefore defined, with a compound of formula XXII,

R^{q2—OH} XXII

wherein R^{q2} is as hereinbefore defined, under standard reaction conditions, for example, in the presence of an acid, such as a hydrogen halide (e.g. HCl).

Intermediate compounds described herein, and derivatives thereof (e.g. protected derivatives), may be commercially available, are known in the literature or may be obtained by conventional synthetic procedures, in accordance with known techniques, from readily available starting materials using appropriate reagents and reaction conditions.

Any of the processes described herein may advantageously be employed in conjunction (i.e. in sequence). For example, processes for the preparation of compounds of formula IIA may consist of, first, a process for the preparation of a compound of formula V as described herein (i.e. comprising reaction of a compound of formula XVII with hydroxylamine, or a salt thereof), followed by a process for the preparation of the compound of formula IIA (i.e. comprising reaction of a compound of formula IV with a compound of formula V so prepared). Further, processes for the preparation of compounds of formula II and/or III (or derivatives thereof) may advantageously be employed in conjunction with the process of the invention.

Substituents on compounds of formula III (or I) or any relevant intermediate compounds to such compounds (or salts, solvates or derivatives thereof), for instance substituents defined by R¹, R², R³, R⁴, or substituents on Y, may be modified one or more times, before, after or during the processes described above by way of methods that are well known to those skilled in the art. Examples of such methods include substitutions, reductions, oxidations, alkylations, acylations, hydrolyses, esterifications, etherifications, halogenations, nitrations, diazotizations or combinations of such methods. In this manner certain compounds of formula I, II or III (or derivative thereof) may be converted to other compounds of formula I, II or III (or derivative), respectively. For instance, a compound of formula IV in which R² represents —NO₂ may be employed (which compound may be better suited to a nucleophilic aromatic substitution reaction of a compound of formula IV with a compound of formula V) to synthesis a compound of formula IIA in which R² is also —NO₂. However, such a —NO₂ group may be reduced to an amino group before or after the process of the invention to form a corresponding compound of formula I in which R² represents amino. Such an amino group may not have been suited to the above-mentioned nucleophilic aromatic substitution reaction, if initially an amino substituted compound of formula IV was deployed. Likewise a compound corresponding to a compound of formula III but in which Y represents aryl or heteroaryl substituted by —NH₂ may be employed in the process of the reaction, but that amino group may be converted to a diazonium salt, and then subsequently to, for example, a —OH group, before or after the process of the reaction.

It is stated herein that specific functional groups may be protected. It will also be appreciated by those skilled in the art that, in the processes described above, other functional groups of intermediate compounds may be, or may need to be, protected by protecting groups.

In any event, functional groups which it is desirable to protect include hydroxy (although certain hydroxy groups in the processes described herein are specifically indicated as being unprotected, i.e. free —OH, derivatives). Suitable protecting groups for hydroxy include trialkylsilyl and diarylalkyl-silyl groups (e.g. tert-butyldimethylsilyl, tert-butyldiphenylsilyl or trimethylsilyl), tetrahydropyranyl and alkylcarbonyl groups (e.g. methyl- and ethylcarbonyl groups). However, most preferred protecting groups for hydroxy include alkylaryl groups, such as optionally substituted benzyl.

The protection and deprotection of functional groups may take place before or after any of the reaction steps described hereinbefore.

Protecting groups may be removed in accordance with techniques which are well known to those skilled in the art and as described hereinafter.

The use of protecting groups is described in “Protective Groups in Organic Chemistry”, edited by J. W. F. McOmie, Plenum Press (1973), and “Protective Groups in Organic Synthesis”, 3^{rd} edition, T. W. Greene & P. G. M. Wutz, Wiley-Interscience (1999).

The skilled person will appreciate that the benzofuran-forming process of the invention may proceed via an O-phenyl oxime intermediate, i.e. a compound of formula XXIV,

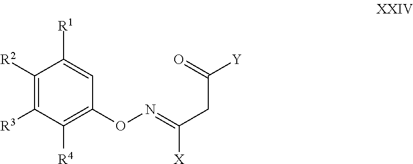

wherein R¹ to R⁴, X and Y are as hereinbefore defined, which intermediate then undergoes a pericyclic rearrangement, ultimately forming a benzofuran ring. It is hereinbefore stated that in an embodiment of the invention, the process of the invention is performed in the absence of an acylating agent. In this instance, when the process of the invention proceeds via an intermediate of formula XXIV, then the phenyl oxime intermediate of formula XXIV does not first react with an acylating reagent to form an N-acyl group at the imino nitrogen (the relevant imino functional group being converted to enamino functional group), for example as depicted by the following compound of formula XXIVA,

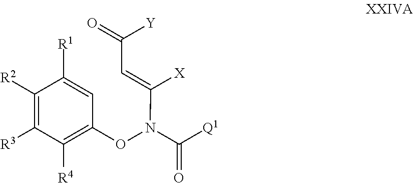

or another enamino equivalent thereof (for example, when X represents an alkyl group, the double bond of the enamino moiety may be adjacent the X group), wherein Q¹ represents, for example, a C₁₋₆ alkyl group optionally substituted by one or more fluoro atoms (so forming, for example a —CF₃ group) and R¹ to R⁴, X and Y are as hereinbefore defined.

Rather, the pericyclic rearrangement of the compound of formula XXIV takes place in the absence of an acylating reagent and hence does not proceed via an intermediate of formula XXIVA. Rather, the pericyclic rearrangement is performed under reaction conditions such as those described herein, for example in the presence of acid, such as a weak organic acid as described herein.

Such an intermediate may be separated (e.g. isolated) in the process of the invention and/or reaction conditions may subsequently be modified. That is, in a first reaction step, a compound of formula II, as hereinbefore defined, may be reacted with a compound of formula III, as hereinbefore defined, to form an intermediate compound of formula XXIV and, in a subsequent reaction step, the intermediate of formula XXIV may undergo reaction (i.e. a pericyclic rearrangement reaction) to form the compound of formula I. Hence, such an embodiment essentially consists of two (e.g. distinct/separate) reaction steps. In such an embodiment, the intermediate compound of formula XXIV may be separated (e.g. extracted, optionally isolated from any impurities, and any solvent optionally removed) from the reaction mixture and/or the subsequent reaction step may be performed under modified reaction conditions (e.g. in the presence of a different, or ‘fresh’, solvent and/or in the presence of additional reagents).

However, advantageously, any intermediate formed in the benzofuran-forming process of the present invention (such as an intermediate of formula XXIV) need not be separated and/or reaction conditions need not be modified in order to promote the benzofuran-forming reaction. In essence, therefore, the reaction may be performed as a “one-pot” procedure. Such a “one-pot” procedure is particularly preferred in the case where compounds of formula I in which Y represents H (and/or compounds of formula I in which R² represents —NO₂) are required and/or desired.

Thus, in particular embodiments of the invention, the reaction is performed without separation (e.g. isolation) of any intermediates. In alternative embodiments of the invention, the reaction is conducted without modification of the reaction conditions.

Where it is stated that the reaction is performed without separation of intermediates, we mean that any intermediate that may be formed by reaction of the starting reagents, is not isolated, e.g. in a purified state (whether or not the intermediate is still in the presence of solvent and/or residual starting materials or other impurities). In this context, we therefore include that the any intermediate is not extracted from the reaction of the starting materials. Where it is stated that the reaction conditions need not be modified, we encompass reactions in which the solvent need not be changed and/or that further reagents need not be added.

In yet another aspect of the invention, there is provided a benzofuran-forming process for the preparation of a compound of formula I as hereinbefore defined which comprises reaction, for example an intramolecular reaction (i.e. pericyclic rearrangement), of a compound of formula XXIV. Such a reaction may be performed in the absence of an acylating reagent, and may for example be performed under the reaction conditions described herein.

The process of the invention (i.e. the benzofuran-forming reaction of a compound of formula II with a compound of formula III) is preferably performed in the presence of an acid, such as a weak organic acid (e.g. formic acid or, preferably, acetic acid) and/or an inorganic acid, such as any suitable mineral acid, or suitable salts thereof (for example, nitric acid, sulfuric acid, or salts thereof, such as sodium hydrogen sulphate, or, more preferably, a hydrogen halide acid, e.g. HBr). Mixtures of acids may also be employed, for instance, a mixture of a weak organic acid and an inorganic acid (e.g. HBr and acetic acid). Further, when an acid is employed, then that acid may be a component of an aqueous solution. By “weak organic acid”, we mean that the organic acid has a pKa (at about 25° C.) of from about 2 to about 6 (e.g. from about 3 to about 5).

The benzofuran-forming process of the invention may be performed in the presence of a suitable solvent, for example water or an organic solvent such as toluene, tetrahydrofuran, diethyl ether, dioxane, dimethylformamide, dimethylsulfoxide, or, preferably an alcohol (such as methanol or ethanol), or mixtures thereof (including biphasic solvent systems, such as a mixture of water and an organic solvent). However, when a weak organic acid is employed (whether it is as the only acid component or as a component of a mixture of acids) in the reaction mixture, then that acid may serve as both the reagent and solvent. In such an instance, advantageously, the separate use of a solvent in the reaction mixture is circumvented (although, as stated above, a mixture of such a organic acid and another suitable solvent, as defined above, may be employed). In particular, weak organic acids that have a relatively low boiling point may serve as the reagent and solvent, for instance those organic acids with a boiling point of less than 150° C. (e.g. formic or, more preferably, acetic acid). When, for instance, a weak organic acid (e.g. that serves as reagent and solvent) is employed, then it may be employed as a solution (e.g. in water or an organic solvent) or, e.g. more preferably, it is employed “neat”. For instance, when acetic acid is employed, then it may be glacial acetic acid.

When a solvent, or a weak organic acid that serves as a solvent, is employed, then it may be present in any suitable volume. However, it is preferred that the concentration of the compound of formula II in the solvent/weak organic acid solvent is from about 0.1 M to about 5 M, preferably from about 0.5 M to about 2 M (e.g. between about 0.6 M and 1.5 M).

In the event that the compounds of formula II and III are added to the reaction mixture at the same time, then the concentration of the reagents in the solvents will be higher (in accordance with the molar ratios of the compounds of formulae II and III in the reaction mixture; see below). However, it is preferred that the compound of formula III is added to the compound of formula II (which latter is preferably already in the presence of a solvent or weak organic acid that serves as a solvent). However, it is particularly preferred that a compound of formula II is added to a compound of formula III (the latter preferably already in the presence of a solvent or weak organic acid that serves as a solvent). Such an order of addition may aid the regioselectivity of the initial intermolecular reaction and/or, in the case where the reaction proceeds via an intermediate compound of formula XXIV, this order of addition may also aid the efficiency of the subsequent intramolecular reaction forming the benzofuran ring.

The benzofuran-forming process of the reaction may be performed at any suitable reaction temperature, for instance at room or elevated temperature. In certain preferred embodiments of the invention, (e.g. when the reaction takes place in the presence of a mixture of a weak organic acid and strong inorganic acid) the reaction may be performed at room temperature (e.g. for a period of time, such as about 6 hours), or, (e.g. when the reaction takes place in the presence of a weak organic acid solvent) the reaction may be performed at elevated temperature (e.g. at above 50° C., such as between about 60° C. to about 80° C.) for a period of time (such as about 3 hours, or, any suitable period of time up to about 25 hours) followed by, if necessary, an increase in reaction temperature (e.g. to at least 80° C., for instance from about 90° C. to about 118° C. (e.g. such as about 110° C., e.g. about 100° C.)), for a period of time (such as any suitable period of time up to about 25 hours, for instance, 22 hours).

The skilled person will appreciate that the temperature may only be increased up to the boiling point of the solvent system (which may comprise a weak organic acid solvent), for instance, when acetic acid is employed, the reaction temperature may only be increased up to about 118° C. Hence, the preferred temperature conditions of the process of the invention are particularly applicable when the process of the reaction is performed in the presence of acetic acid. However, when the process of the reaction is performed in the presence of other weak organic acids (or otherwise another suitable solvent), such as formic acid, the skilled person will appreciate that the preferred reaction temperature conditions referred to herein may be varied, for example in accordance with differing boiling points.

The benzofuran-forming process of the invention may also be conducted under conditions that provide an alternative to typical reaction conditions where elevated temperatures are necessary and/or desired. For instance, microwave irradiation conditions may be employed. By ‘microwave irradiation conditions’, we include reactions in which such conditions promote a thermally induced reaction (for instance at elevated temperature as hereinbefore described) and/or in which such conditions promote a non-thermally induced reaction (i.e. the reaction is essentially induced by the microwaves). Hence, such reaction conditions are not necessarily accompanied by an increase in temperature. The skilled person will appreciate (and be able to non-inventively determine) that the length of reaction time may be altered (e.g. reduced) when employing such reaction conditions.

The benzofuran-forming process of the invention may also be conducted under pressure, for instance, under a pressure greater than that of normal atmospheric pressure, for example, at a pressure of up to about 5 or 6 bars. Again, the skilled person will appreciate (and be able to non-inventively determine) that the length of reaction time may be altered (e.g. appropriately reduced) when employing such reaction conditions.

The benzofuran-forming process of the invention may be performed in the presence of any quantity of each of the compounds of formulae II and III. However, it is preferably performed in the presence of compounds of formulae II and III that are in a molar ratio of from about 3:2 to about 2:3, and most preferably in a molar ratio of from about 1.1:1 to about 1:1.1 (e.g. about 1:1).

Preferred compounds of formula I that may be prepared by the process of the invention include those in which:
R¹, R², R³ and R⁴ independently represent hydrogen, halo, —NO₂, —CN, —C(O)₂R^{x1}, —N(R^{x6})R^{x7} or —N(R^{x10})S(O)₂R^{x11};
X represents C₁₋₄ alkyl (optionally substituted by one or more fluoro atoms; but preferably, unsubstituted), for example C₄ alkyl, such 1-methylpropyl, or, most preferably, butyl (especially n-butyl);
Y represents phenyl substituted by one —OH group in the 2-, 3- or, preferably, in the 4-position;
R^{x1}R^{x2}, R^{x3}, R^{x6}, R^{x7}, R^{x8}, R⁹ and R^{x10} independently represent hydrogen or C₁₋₄ alkyl optionally substituted by one or more halo (e.g. fluoro) atoms;
R^{x4}, R^{x5}, R^{x11} and R¹² independently represent C₁₋₄ alkyl optionally substituted by one or more halo (e.g. fluoro) atoms.

Further preferred compounds of formula I that may be prepared by the process of the invention include those in which:
any three of R¹, R², R³ and R⁴ (preferably R¹, R³ and R⁴) represent hydrogen;
any one of R¹, R², R³ and R⁴ (preferably R²) represents a substituent selected from halo, —CN, —C(O)₂R^{x1}, preferably, —N(R^{x10})S(O)₂R^{x11} or, more preferably, —NO₂ or —N(R^{x6})R^{x7} (e.g. —NO₂);
R^{x1} represents H or C₁₋₃ alkyl (e.g. propyl, such as isopropyl);
R^{x6}, R^{x7} and R^{x10} independently represent hydrogen;
R^{x11} represents C₁₋₂ alkyl (e.g. methyl).

Reactions to produce such compounds of formula I have the additional advantage that, when 3-aroyl substituted benzofurans are required, a (disadvantageous) Friedel-Crafts acylation step on a 3-unsubstituted benzofuran is circumvented. Further advantages associated with this process of the invention are that compounds of formula I may be produced in higher yields as the reaction may proceed in a more regioselective manner than corresponding reactions. Despite the fact that the compound of formula III contains two carbonyl moieties, the reaction with the compound of formula II proceeds in a highly regioselective manner, favouring the carbonyl adjacent to (or α- to) the group defined by X (in the initial step condensation reaction between the hydroxylamino moiety of the compound of formula II and the relevant carbonyl group). Surprisingly, this regioselectivity is greater than 90:10 (e.g. 95:5), and selectivities of 99:1 have been achieved.

As stated hereinbefore, it is preferred that compounds of formula I obtained via the benzofuran-forming process of the invention are ones in which R² represents —NO₂. The formation of compounds of formula I in which R² is —NO₂ normally proceeds via a reaction of a chlorophenyl group with a hydroxy-imine (e.g. 2-hexanone oxime), which is the conventional manner of performing this reaction.

When such compounds of the invention (prepared by processes of the invention described herein) are desired and/or required (for example as an intermediate in the synthesis of Dronedarone), it is particularly advantageous that the process of the invention proceeds when the relevant —OH group is unprotected. For instance, processes described in the prior art (e.g. in U.S. Pat. No. 5,223,510, U.S. Pat. No. 5,854,282 and PCT/EP2007/004984), which relate to the Friedel-Crafts acylation of 3-unsubstituted benzofurans, all result in the formation of 3-(4-methoxybenzoyl)benzofurans. Such intermediates may be employed in the synthesis of Dronedarone, but the methoxy group has to be ‘deprotected’, i.e. the methyl group has to be cleaved from the methyl aryl ether. Such cleavage conditions may also involve metal halide catalysts, such as group III metal halide catalyst, such as BBr₃ and AlCl₃ (which are disadvantageous in process chemistry for reasons mentioned herein; for example as toxic by-products may be formed, e.g. chloromethane, when AlCl₃ is employed). Hence, given that when compounds of formula I in which Y represents phenyl substituted (e.g. in the para-position) with —OH are prepared, such methyl aryl ether cleavage is circumvented, this embodiment of the invention is particularly preferred. Hence, there are several environmental benefits associated with the process of the invention, and particularly with certain embodiments of the process of the invention.

The compounds of formula I obtained by the process of the invention may be separated and/or isolated by standard techniques, for instance by chromatography, crystallisation, evaporation of solvents and/or by filtration.

Advantageously, the process of the invention further comprises the additional step of crystallisation of the compound of formula I from a solution, wherein the solvent is preferably, a non-halogenated solvent. Such a crystallisation may be performed by the addition of a solvent to the reaction mixture of the process of the invention that provides for a compound of formula I (e.g. without prior separation, e.g. isolation, (e.g. by extraction) of the compound of formula I) or, such a crystallisation may be performed after the compound of formula I is separated (e.g. by extraction, optionally followed by removal of solvent) or isolated.

Preferably, the crystallisation mixture/solution (which, in this context, includes a compound of formula I in the reaction mixture after the process of the invention but prior to separation, as well as a compound of formula I that is separated and to which a solvent is then added) is cooled after the addition of the solvent. Conveniently, the mixture is cooled to between about −5 and about 15° C. (for example the optimal temperatures employed are between about +5 and about 15° C.). A preferred ‘crystallisation’ temperature is about −5° C. (minus five degrees Celsius). The mixture may be cooled using any suitable means, for example ice-baths or cooling systems well known to those skilled in the art and include, for example, heat exchangers.

The ‘crystallisation’ solvent may also be used to wash the crystallised product, which solvent is preferably pre-cooled. Possible temperatures to which the solvent may be pre-cooled are between about −5° C. to about 5° C. (or, alternatively, the temperature may be between about +5 and about 15° C.). If there is no pre-cooling of the washing solvent, yield may drop. The most preferred temperature is about −5° C.

The ‘crystallisation’ solvent is preferably a non-halogenated one, e.g. water or it may be an alcohol, such as methanol ethanol, iso-propanol and 1-propanol. The most preferred ‘crystallisation’ solvent may be methanol. Other preferred crystallisation solvents that may be mentioned include weak organic acids, for example, carboxylic acids (such as butanoic acid, propanoic acid, preferably, formic acid or, more preferably, acetic acid). Such weak organic acids may be mixed with water to form crystallisation co-solvents. When the crystallisation consists of the addition of solvent to a reaction mixture, then that solvent may be water.

It should be appreciated that the purified compound of formula I so formed by the process of the invention may also contain materials other than those specified above.

This product may be further purified using any suitable separation/purification technique or combination of techniques including further crystallisation, distillation, phase separation, adsorption, e.g. using molecular sieves and/or activated carbon, and scrubbing.

In a further aspect of the invention there is provided a process for preparing Dronedarone:

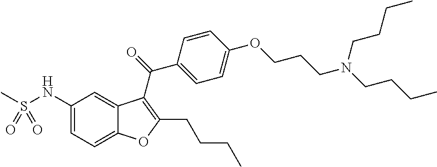

(or a salt, e.g. a hydrochloride salt, thereof), which process is characterised in that it includes as a process step a process as described herein (for instance, a process for the preparation of 2-butyl-3-(4-hydroxybenzoyl)-5-nitrobenzofuran).

Hence, there is provided a process for the preparation of Dronedarone, or a salt thereof, comprising a process for the preparation of a compound of formula I (e.g. a process for the preparation of 2-butyl-3-(4-hydroxybenzoyl)-5-nitrobenzofuran) as described herein, followed by, if necessary/required:
1) conversion of the nitro (—NO₂) group to a methylsulfonylamino (—NHS(O)₂CH₃) group (for example via the conversion of the nitro group to an amino (—NH₂) group, followed by reaction with CH₃—S(O)₂-L^{a}, in which L^{a} represent halo, and preferably chloro);
2) conversion of the —OH group to the relevant oxy-alkylaminoalkyl (e.g. —O—(CH₂)₃—N(C₄H₉)₂) group;
3) if necessary/required, conversion of any free base of Dronedarone so formed to a salt (such as a hydrochloride salt).

Such steps are standard steps known to the skilled person, and the steps may be performed in accordance with techniques described in the prior art, such as those references disclosed herein. For example, Dronedarone (or salts thereof) may be prepared from the relevant compounds of formula I using any standard route of synthesising derivatives of benzofuran, such as those described in U.S. Pat. No. 5,223,510. The skilled person will appreciate that the individual steps of the conversions (e.g. those outlined by steps (1) and (2) above) may be performed in any suitable order.

### Step (2)

For example, when the compound of formula I is 2-butyl-3-(4-hydroxybenzoyl)-5-nitrobenzofuran, then such a compound may be reacted as set out by step (2) above, which reaction may be performed in the presence of a compound of formula XXV,

L^{1a1}-(CH₂)₃—N(n-butyl)₂ XXV

wherein L^{1a1} is a suitable leaving group, such as a sulfonate group (e.g. a triflate or sulfonate), iodo, bromo or, preferably, chloro, under standard alkylation reaction conditions, for example such as those described in U.S. Pat. No. 5,223,510 (see Example 1(e)), to form a Dronedarone intermediate compound of formula XXVI.

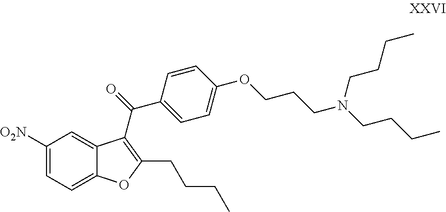

Alternatively, step (2) may be performed in two distinct steps, for example, by reaction of 2-butyl-3-(4-hydroxybenzoyl)-5-nitrobenzofuran with a compound of formula XXVIA,

L^{1a1}-(CH₂)₃-L^{1a1} XXVIA

wherein each L^{1a1} independently represents a suitable leaving group, such as iodo, chloro or, preferably, bromo, so forming a Dronedarone intermediate of formula XXVIB,

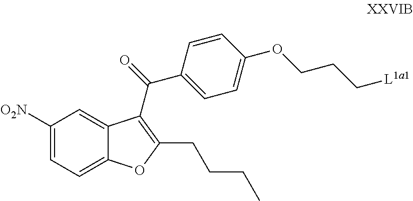

wherein L^{1a1} is as hereinbefore defined (and is preferably bromo), which intermediate may then be reacted with HN(n-butyl)₂ (di-n-butylamine) to form a Dronedarone intermediate of formula XXVI, for example under reaction conditions such as those described in Chinese patent publication number CN 101153012).

Step (1)

The intermediate compound of formula XXVI may then be reacted as set out by step (1) above, which may consist of distinct sub-steps:
(i) reduction of the —NO₂ group to a —NH₂ group, under standard reaction conditions, for example such as those described in U.S. Pat. No. 5,223,510 (see Example 1(f)) or in WO 02/48132, for example hydrogenation in the presence of H₂ (e.g. a hydrogen atmosphere or nascent hydrogen, e.g. ammonium formate) and a precious metal catalyst (e.g. PtO₂ or Pd/C), in the presence of an appropriate solvent (e.g. an alcohol, e.g. ethanol), thereby forming an intermediate compound of formula XXVI,

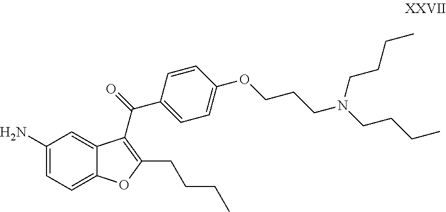

(ii) the Dronedarone intermediate compound of formula XXVII may then be mesylated by reaction with a compound of formula XXVIII,

H₃C—S(O)₂-L^{1a2} XXVIII

wherein L^{1a2} represents a suitable leaving group, such as bromo, iodo or, preferably, chloro, under reaction conditions such as those described in U.S. Pat. No. 5,223,510 (Example 3(a)).

### Step (3)

As stated above, Dronedarone may be converted into a salt, such as a hydrochloride salt, for example as described in U.S. Pat. No. 5,223,510 (see Example 3(b)), for example by bringing into association Dronedarone and HCl in ether, or as described in U.S. Pat. No. 6,828,448 (see Examples, such as Example 4), for example by bringing into association Dronedarone, hydrochloric acid (e.g. about 30-40%) and an alcoholic solvent, such as isopropanol.

As stated above the above steps may be performed in any feasible order. Hence, 2-butyl-3-(4-hydroxybenzoyl)-5-nitrobenzofuran may first be reacted as set out in step (1), followed by the reaction(s) as set out in step (2). The preparation of Dronedarone may therefore proceed via the following intermediate compounds of formulae XXIX and XXX (step (1)),

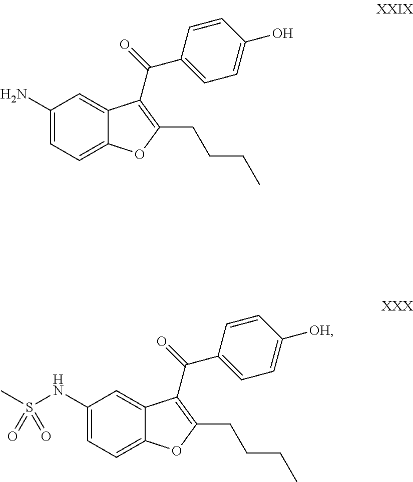

and, may also proceed via the intermediate compound of formula XXXI (step (2), when performed as a two-step process),

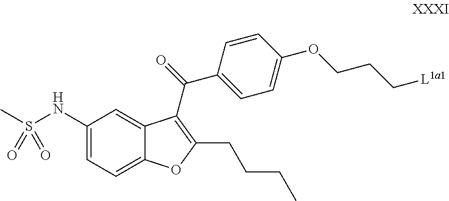

wherein L^{1a1} is as hereinbefore defined.

The skilled person will appreciate that the intermediate compounds of formulae XXVI, XXVIB, XXVII, XXIX, XXX and XXXI may also be compounds of formula I. Hence, the conversion of such compounds of formula I (which may be prepared directly from the process of the invention) may not require all of the process steps (or sub-process steps) outlined above (i.e. steps (1), (2) and (3)) in order to provide Dronedarone, or a salt (e.g. a HCl salt) thereof. In such instance, it is immediately clear to the skilled person which of the above-mentioned steps are required for the appropriate conversions.

There is further provided a process for the preparation of an intermediate of Dronedarone (or a salt thereof, e.g. a hydrochloride salt), which process comprises a process step as hereinbefore described followed by one or more process steps that lead to the formation of Dronedarone, or a salt thereof. For example, such further process steps may include any one or more of the process steps disclosed in steps (1), (2) and (3) above, in any feasible order (thereby forming an intermediate of formula XXVI, XXVIB, XXVII, XXIX, XXX or XXXI). The skilled person will appreciate that steps (1), (2) and (3) above may each require multiple separate reaction steps for the relevant conversion to be effected.

The processes described herein may be operated as a batch process or operated as a continuous process and may be conducted on any scale.

In general, the processes described herein, may have the advantage that the compounds of formula I may be produced in a manner that utilises fewer reagents and/or solvents, and/or requires fewer reaction steps (e.g. distinct/separate reaction steps) compared to processes disclosed in the prior art.

The process of the invention may also have the advantage that the compound of formula I is produced in higher yield, in higher purity, in higher selectivity (e.g. higher regioselectivity), in less time, in a more convenient (i.e. easy to handle) form, from more convenient (i.e. easy to handle) precursors, at a lower cost and/or with less usage and/or wastage of materials (including reagents and solvents) compared to the procedures disclosed in the prior art. Furthermore, there may be several environmental benefits of the process of the invention, such as the circumvention of the use of halogenated solvents (e.g. when avoiding the need to perform a Friedel-Crafts reaction or a deprotection of e.g. a —OCH₃ group, which may be required for certain steps performed by processes in the prior art, to a —OH group).

The following examples are merely illustrative examples of the processes of the invention described herein.

All equipment, reagents and solvents used were standard laboratory equipment, e.g. glassware, heating apparatus and HPLC apparatus.

Non-limiting examples, with reference to the following figures are described:

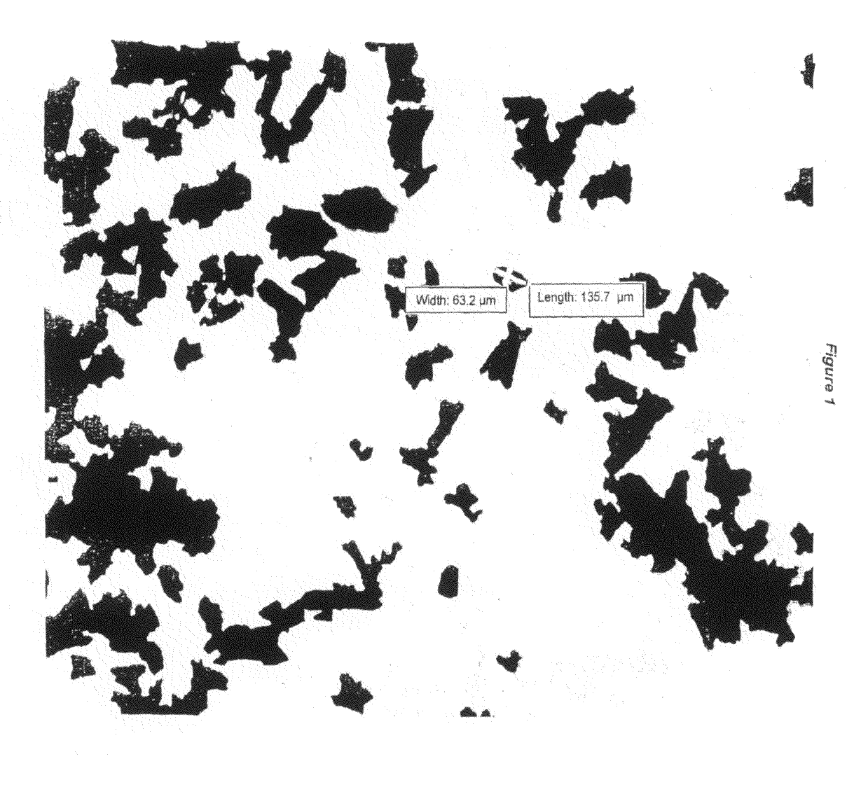

FIG. 1: picture of particle size of 1-(4-hydroxyphenyl) heptane-1,3-dione produced by Example A (Example 1, (b)) of international patent application WO 2009/044143 (the measurement is 135.7 μM (length)×63.2 μM (width).

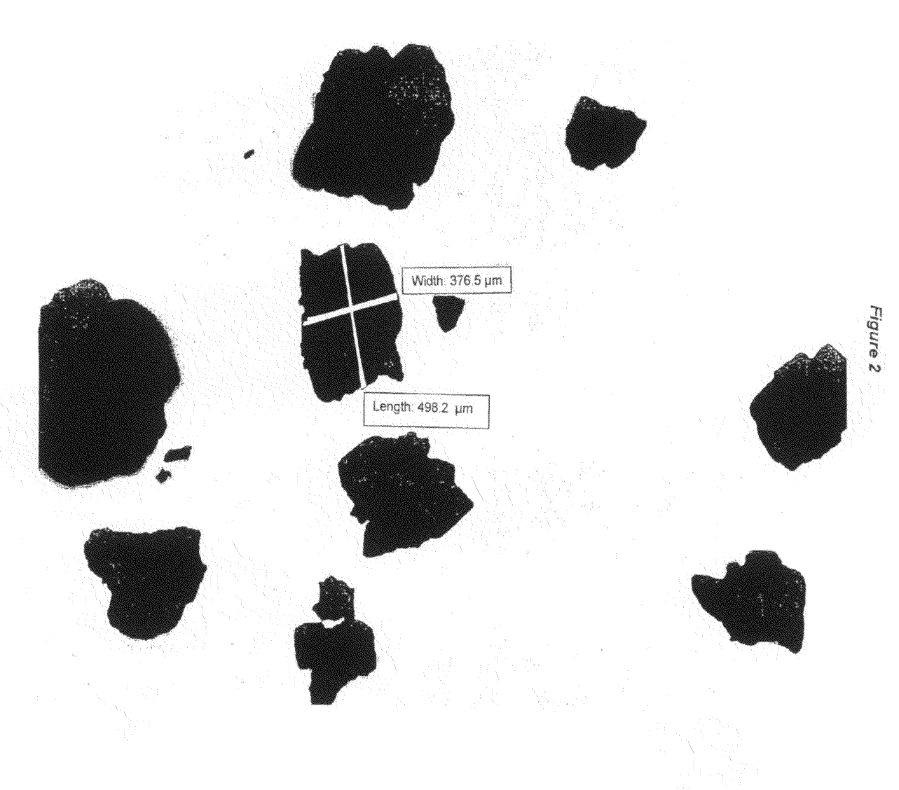

FIG. 2: picture of particle size of 1-(4-hydroxyphenyl) heptane-1,3-dione as produced by processes of the invention described herein (e.g. Example 1 (a) below; the measurement is 498.2 μM (length)×376.5 μM (width)).

### EXAMPLE 1

### (a) 1-(4-hydroxyphenyl) heptane-1,3-dione

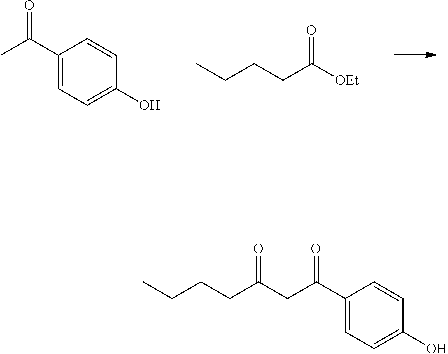

Sodium t-butoxide, 180.5 g, 1.878 mol, is mixed and stirred with 378 ml THF. A mixture of 4-hydroxy acetophenone, 85.3 g, 0.626 mol and ethyl valerate, 81.5 g, 0.626 mol in 56 ml THF is heated to ca 45° C. and the clear solution is added to the sodium t-butoxide/THF mixture. The mixture is heated to reflux temperature (ca 68° C.) and stirred for 6 h. The temperature is adjusted to ca 60° C. and the viscous mixture is quenched by addition to a solution of 120 g acetic acid in 294 ml water. THF and other volatiles are stripped and the residual emulsion is extracted with 146 ml toluene. After separation of the water phase, the residue is concentrated under vacuum and the product crystallised from a mixture of 130 ml acetic acid and 138 ml water. The product is isolated by filtration and the filter cake washed with 20% acetic acid followed by water. The wet product is dried under vacuum to afford 93.1 g, 0.423 mol 1-(4-hydroxyphenyl) heptane-1,3-dione. Yield 67.5%.

### (b) 1-(4-hydroxyphenyl)-4-methylhexane-1,3-dione

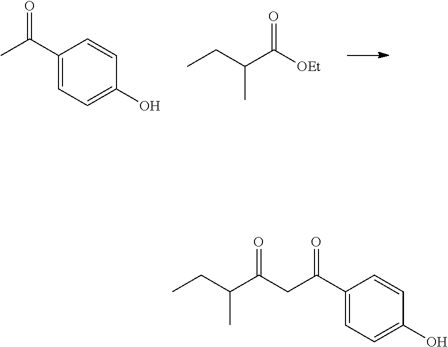

4-Hydroxy acetophenone, 29.0 g, 0.213 mol and ethyl 2-methylbutyrate, 27.7 g, 0.213 mol, are dissolved in 80 ml THF. The solution is added to a slurry of 63.3 g, 0.659 mol, sodium tert-butoxide in 80 ml THF. The resulting mixture is heated to reflux (70° C.) and stirred for 70 hours. The dark solution is quenched by adding it to a mixture of 55 ml 37% HCl and 100 ml water. Volatiles are stripped under vacuum and to the residual is added 50 ml toluene and 40 ml water. The water phase is separated and the toluene phase is washed with 50 g 10% NaCl. The water phase is separated and the toluene stripped under vacuum at 75° C. leaving 43.5 g of a red oil. Attempted crystallization from acetic acid/water was unsuccessful. Purity (HPLC) 90%. Yield: 0.9×43.5=39.2 g, 0.178 mol, 83.5%

### (c) 1-(4-hydroxyphenyl)heptane-1,3-dione

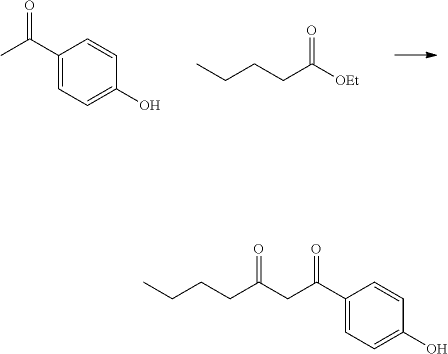

To a solution of 4-hydroxy acetophenone, 13.6 g, 0.10 mol, in 74 ml ethyl valerate. is added sodium tert-butoxide, 29.7 g, 0.31 mol, in portions. The formed slurry is heated to 82° C. and stirred for 4 hours after which the mixture is quenched by addition to a solution of 2 ml acetic acid in 47 ml water. The product-containing lower water phase is separated and treated with acetic acid, 16 ml, to reach pH 4. The upper oily phase is separated and diluted with 20 ml acetic acid and 2.3 g water. The mixture is cooled and crystals starts to separate at 20° C. Cooling is continued to 5° C. 19 ml Water is added over 25 minutes followed by stirring for 20 minutes and then the product is isolated by filtration, washed with 23.5 g 20% acetic acid followed by 23.5 g water. Drying at room temperature in an air stream afforded 14.6 g 1-(4-hydroxyphenyl)heptane-1,3-dione. Purity (HPLC)<99.8%, yield 65%. The upper phase from the quench is diluted with 30 ml toluene and a small water phase is separated. Concentration of the organic phase followed by distillation afforded crude ethyl valerate, 48% of theoretic recovery.

### (d) 1-(4-hydroxyphenyl)heptane-1,3-dione

To sodium tert-butoxide (190 g, 1.98 mol) is added 459 ml THF. The mixture is stirred until only a thin slurry remains. The mixture is added to a suspension of 4-hydroxyacetophenone (89.5 g, 0.657 mol) in ethylvalerate (85.6 g, 0.657 mol). The mixture is heated to 70-73° C. and stirred at this temperature until conversion of 4-hydroxyacetophenone is >90% (ca 15 h), The mixture is cooled to ca 60° C. and added to a mixture of 308 g water and 126 g acetic acid (2.1 mol). The contents are heated and THF and other volatiles are stripped at atmospheric pressure until the temperature in the reactor reaches 102° C. The batch is cooled to ca 75° C. and the lower water phase is separated and discarded. The reactor content is heated under vacuum in order to strip residual ethylvalerate. The operation is interrupted when the liquid temperature is z 110° C. at a pressure 50 mbar. The remaining product oil is diluted with 142 g acetic acid followed by 61 g water and the mixture is cooled to 25-28° C. and stirred until a thick slurry has formed. The slurry is cooled to ca −12° C. and 43 ml water added over ca 45 minutes followed by stirring at about −12° C. for 60 minutes. The product is isolated by filtration and the filter cake washed with 150 g 20% acetic acid followed by 150 g water. Drying under vacuum at 50° C. gives 1-(4-hydroxyphenyl)heptane-1,3-dione, 104 g, 0.472 mol. Purity HPLC: 99.9%. Yield: 72%.

### EXAMPLE 2

### 2-Butyl-3-(4-hydroxybenzoyl)-5-nitrobenzofuran

O-4-nitrophenylhydroxylamine (1.0 g), was suspended in acetic acid (10 ml) and 1-(4-hydroxyphenyl)-heptane-1,3-dione (1.36 g; prepared in accordance with Example 1(a) or Example 1(c) above) was added. The mixture was stirred for 3 h at 70° C. and then at 100° C. for an additional 22 h. The mixture was cooled to room temperature and the solvent evaporated under vacuum. Yield 80% of 2-Butyl-3-(4-hydroxybenzoyl)-5-nitrobenzofuran.

### EXAMPLE 3

### Synthesis of Dronedarone

Dronedarone is synthesised using standard synthetic processes described in the prior art (and referenced herein) incorporating any of the processes described herein, for example the process to the intermediates 2-butyl-3-(4-hydroxybenzoyl)-5-nitrobenzofuran described in Example 2 or the process to the intermediate described in Example 1 (e.g. Examples 1(a) or Example 1(c)). Dronedarone can be made from these intermediates using any standard routes for converting a nitro (—NO₂) group to a methylsulfonylamino (—NHS(O)₂CH₃) group (for example via an amino (—NH₂) group) and converting a —OH (or —OCH₃) group to any relevant oxy-alkylaminoalkyl (e.g. —O—(CH₂)₃—N(C₄H₉)₂) group. Further, salts (such as hydrochloride salts) of the relevant compounds may also be prepared. Such steps are standard steps known to the skilled person, and the steps may be performed in accordance with techniques described in the prior art, such as those references disclosed herein.

### EXAMPLE 4

### Method A

Ethyl N-(4-nitrophenoxy)acetimidate

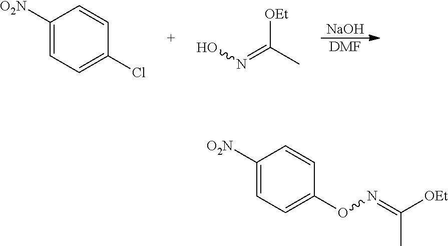

4-Chloronitrobenzene, 136.2 g, and 111.4 g ethyl N-hydroxyacetimidate are dissolved in 216 ml DMF. The temperature is adjusted to 30° C. and 41.6 g solid NaOH is added in 8 portions keeping the temperature at 30-35° C. After one hour the temperature is adjusted to 40-45° C. and the mixture stirred for 1.5 hours. Cooling is applied and 520 ml water is fed at such a rate as to keep the temperature at ca 40° C. The slurry formed is cooled to 17° C. and filtered. The filter cake is washed with 175 ml ethanol/water 90/10 (V/V) followed by 175 ml water. Wet product, 214.5 g, corresponding to 192 g dry ethyl N-(4-nitrophenoxy)acetimidate is isolated. Yield 98.5%.

### Method B

Ethyl N-(4-nitrophenoxy)acetimidate

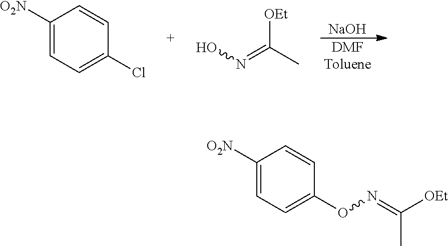

To a solution of 549 g ethyl N-hydroxy acetimidate in 976 g toluene is added 1267 g DMF, 39.9 g Aliquat 336 and 799 g 4-chloronitrobenzene. The temperature is adjusted to 30° C. and 223 g solid NaOH is added in portions of 25-30 g every 10-15 minutes. When addition is complete, the jacket temperature is set to 40° C. and the mixture stirred until reaction is complete, 3-4 h. The jacket temperature is adjusted to 50° C. and ca 80% of the toluene stripped at reduced pressure. 3040 g Water is added keeping the temperature at max 45° C. The formed slurry is efficiently agitated and the residual toluene stripped at reduced pressure. After cooling to 15° C. the product is filtered and washed with 1080 g EtOH/water 90/10 (V/V) followed by 1080 g water. Wet product, 1188 g, corresponding to 1080 g dry ethyl N-(4-nitrophenoxy)acetimidate is obtained. Yield 95%.

### Method C

### (a) O-(4-Nitrophenyl)hydroxylamine

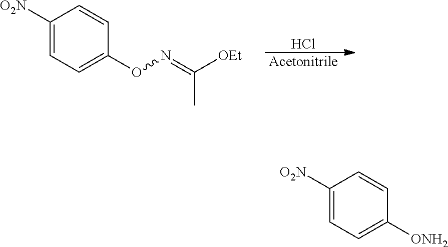

Wet ethyl N-(4-nitrophenoxy)acetimidate, 781 g (dry weight) is dissolved in 2100 g acetonitrile and the temperature adjusted to ca 25° C. 515 g 37% hydrochloric acid is added at such rate as to keep the temperature below 30° C. The mixture is stirred at 25-30° C. until the reaction is complete, ca 2 h. Then 2090 g of 12% NaOH(aq) is added at 25-30° C. and the mixture stirred for ca 30 minutes. Vacuum is applied and ca 85% of the acetonitrile stripped at 100 mbar and a jacket temperature of 50° C. (inner temperature 25-30° C.). Water, 2090 g, is added and the slurry stirred for 60 minutes. The product is filtered and washed with 505 g water followed by drying under vacuum at 40° C. O-(4-Nitrophenyl)hydroxylamine, 560 g, is obtained. Yield 94%.

### (b) O-(4-Nitrophenyl)hydroxylamine

240 g Water-moist Ethyl-N-(4-nitrophenoxy) acetimidate, containing 181 g, 0.807 mol of product (when dry) was added to 397 g 37% hydrochloric acid (5 eq) in portions over 50 minutes, keeping the temperature at 25-32° C. Analysis (HPLC) after 60 minutes showed a conversion of 99.9%. The slurry was diluted with 37 ml water and then neutralised with 580 g 33% NaOH keeping the temperature below 33° C. The slurry was then cooled to 24° C., filtered, and the filter cake washed with 210 ml water. Drying afforded 124.5 g 0-4-nitrophenyl hydroxylamine. Assay (NMR) 99.8%, chromatographic purity (HPLC) 99.4 area %. Yield 99.9%

### Method D

1-(4-Hydroxyphenyl)-1,3-heptandione

The title compound was prepared in accordance with the procedure described in Example 1 (e.g. Example (1)(a) and Example 1(c)).

### Method E

2-Butyl-3-(4-hydroxybenzoyl)-5-nitrobenzofuran

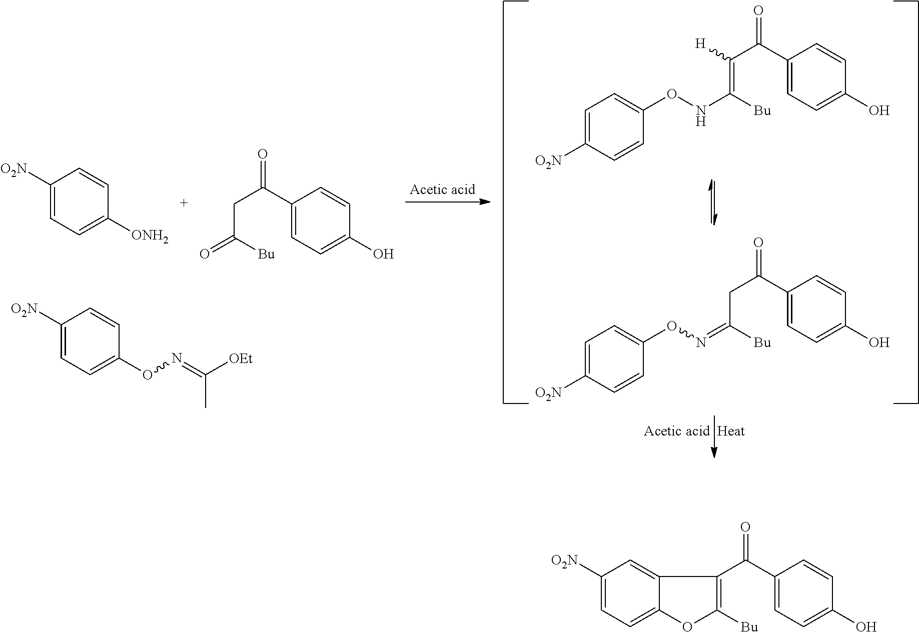

1-(4-hydroxyphenyl)-1,3-heptandione (prepared in accordance with the process of the invention; see Example 1(a) and Example 1(c)), 697 g, is dissolved in 2532 g acetic acid. O-(4-Nitrophenyl)hydroxylamine (see Method C, reactions (a) and/or (b)), 488 g, is added in portions at ca 20° C. The formed slurry is diluted with 739 g acetic acid and the mixture heated to 115° C. and stirred for 3 h. The dark solution is cooled and 1635 g water is added keeping the temperature at 70-80° C. The temperature is adjusted to 60° C. and seeding crystals are added. When crystallisation has started, the slurry is cooled to 4° C., filtered and washed with 870 g of 67% aqueous acetic acid followed by 580 g water. Drying at reduced pressure at 70° C. gives 736 g 2-butyl-3-(4-hydroxybenzoyl)-5-nitrobenzofuran. Yield 69%.

### Method F

1-(4-Hydroxyphenyl)heptane-1,3-dione-3-[O-(4-nitrophenyl)oxime]

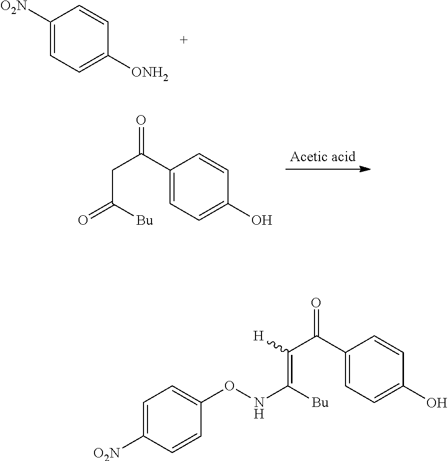

1-(4-Hydroxyphenyl)-1,3-heptandione (prepared in accordance with the process of the invention; see Example 1(a) and Example 1(c)), 1121 g, is dissolved in 4070 g acetic acid. O-(4-Nitrophenyl)hydroxylamine (see Method C, reactions (a) and/or (b)), 784 g, is added in portions keeping the temperature at ca 20° C. The formed slurry is stirred for 3 h, cooled to 15° C., filtered and washed with 1590 g acetic acid. 1944 g wet cake corresponding to 1596 g dry 1-(4-hydroxyphenyl)heptane-1,3-dione-3-[O-(4-nitrophenyl)oxime] is obtained. Yield 88%.

### Method G

2-Butyl-3-(4-hydroxybenzoyl)-5-nitrobenzofuran.

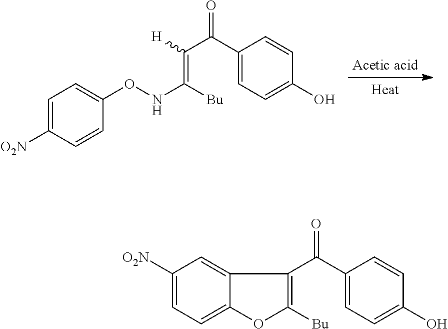

The wet 1-(4-hydroxyphenyl)heptane-1,3-dione-3-[O-(4-nitrophenyl)oxime], 1944 g, obtained in Method F is slurried in 4900 g acetic acid. The slurry is heated to 115° C. and stirred for 3 h. The dark solution formed is cooled and 2630 g water is added keeping the temperature at 70-80° C. The temperature is adjusted to 60° C. and seeding crystals are added. When crystallisation has started, the slurry is cooled to 4° C., filtered and washed with 1400 g of 67% aqueous acetic acid followed by 930 g water. Drying at reduced pressure at 70° C. gives 1182 g 2-butyl-3-(4-hydroxybenzoyl)-5-nitrobenzofuran. Yield 78%.

### Method H—Synthesis of Dronedarone

Dronedarone is synthesised using standard synthetic processes described in the prior art (and referenced herein) incorporating any of the processes described herein, for example the processes to the intermediates 2-butyl-3-(4-hydroxybenzoyl)-5-nitrobenzofuran and 1-(4-hydroxyphenyl)-1,3-heptandione described in the examples above. Dronedarone can be made from these intermediates using any standard routes for converting a nitro (—NO₂) group to a methylsulfonylamino (—NHS(O)₂CH₃) group (for example via an amino (—NH₂) group) and converting a —OH (or —OCH₃) group to any relevant oxy-alkylaminoalkyl (e.g. —O—(CH₂)₃—N(C₄H₉)₂) group. Further, salts (such as hydrochloride salts) of the relevant compounds may also be prepared. Such steps are standard steps known to the skilled person, and the steps may be performed in accordance with techniques described in the prior art, such as those references disclosed herein.

### EXAMPLE 5

Dronedarone may be formulated into a pharmaceutically acceptable formulation using standard procedures, for example to form the product marketed under the brand name, Multaq®.

For example, there is provided a process for preparing a pharmaceutical formulation comprising Dronedarone, or a salt thereof (e.g. a hydrochloride salt), which process is characterised in that it includes as a process step a process as hereinbefore defined. The skilled person will know what such pharmaceutical formulations will comprise/consist of (e.g. a mixture of active ingredient (i.e. Dronedarone or a salt thereof) and pharmaceutically acceptable excipient, adjuvant, diluent and/or carrier).

There is further provided a process for the preparation of a pharmaceutical formulation comprising Dronedarone (or a salt thereof, e.g. a hydrochloride salt; which formulation may be Multaq®), which process comprises bringing into association Dronedarone, or a pharmaceutically acceptable salt thereof (which may be formed by a process as hereinbefore described), with (a) pharmaceutically acceptable excipient(s), adjuvant(s), diluent(s) and/or carrier(s).

There is further provided a process for the preparation of a pharmaceutical formulation comprising Dronedarone (or a salt thereof, e.g. a hydrochloride salt) as described in the art (for example in U.S. Pat. No. 5,985,915 (see Example 3), US 2004/0044070 (see Examples 1 to 5), U.S. Pat. No. 7,323,439, US 2008/0139645 and/or CN 101152154), which process comprises bringing into association Dronedarone (or a salt thereof, e.g. a hydrochloride salt), with the other ingredients of the relevant formulations. For example, Dronedarone hydrochloride may be brought into association with: maize starch, talc, anhydrous colloidal silica, magnesium stearate and lactose (see Example 3 of U.S. Pat. No. 5,985,915); mannitol, anhydrous sodium dihydrogen phosphate and, optionally, water (see Example 5 of US 5,985,915); hydroxypropyl-β-cyclodextrin, monosodium phosphate dehydrate and mannitol (see Example 1 of US 2004/0044070); hydroxypropyl-β-cyclodextrin, anhydrous sodium dihydrogen phosphate, mannitol and, optionally, water (see Examples 2 and 3 of US 2004/0044070); mixture of methylated derivatives of β-cyclodextrin, mannitol and, optionally, water (see Example 4 of US 2004/0044070). The formulations described may be oral tablet forms or injectable forms (e.g. US 2004/0044070 may describe injectable forms).

In particular, there may be further provided a process for the preparation of a pharmaceutical formulation, comprising bringing into association Dronedarone (or a salt thereof; prepared in accordance with the processes described herein), with a pharmaceutically acceptable non-ionic hydrophilic surfactant selected from poloxamers (e.g. poloxamer 407; Synperonic® PE/F127), optionally in combination with one or more pharmaceutical excipients, for example as described in U.S. Pat. No. 7,323,493. For example, Dronedarone hydrochloride may be brought into association with: methylhydroxypropylcellulose, lactose monohydrate, modified corn starch, polyvinylpyrrolidone, Synperonic® PE/F127 and, optionally, any one or more of anhydrous colloidal silica, magnesium stearate and water (see e.g. Tablet A and Examples 1 to 3 of U.S. Pat. No. 7,323,493); modified corn starch, lactose monohydrate, talc, anhydrous colloidal silica and magnesium stearate (see e.g. gelatin capsule of U.S. Pat. No. 7,323,493); microcrystalline cellulose, anhydrous colloidal silica, anhydrous lactose, polyvinylpyrrolidone, Synperonic® PE/F127 and, optionally, one or more of macrogol 6000 and magnesium stearate (see Examples 4 to 6 of U.S. Pat. No. 7,323,493); microcrystalline cellulose, corn starch, polyvinylpyrrolidone, Synperonic® PE/F127, anhydrous colloidal silica, magnesium stearate and lactose monohydrate (see Examples 7 and 8 of U.S. Pat. No. 7,323,493). The skilled person will appreciate that for example in the above-mentioned list of ingredients, every single ingredient need not be present in the formulation (and hence, the process for preparing the formulation may comprise bringing Dronedarone into association with only some of the ingredients mentioned above). Further, where an ingredient is mentioned, the skilled person will appreciate that it may be replaced by another equivalent or similar ingredient that serves the same function (for example Synperonic® PE/F127 may be replaced by another suitable surfactant and methylhydroxypropylcellulose and corn starch may be replaced by another ingredient, such as a suitable disintegrating agent or bioadhesion promoting agent, etc).

When a pharmaceutical formulation is referred to herein, it includes a formulation in an appropriate dosage form for intake (e.g. in a tablet form or an injectable form). Hence, any process mentioned herein that relates to a process for the preparation of a pharmaceutical formulation comprising Dronedarone, or a salt thereof, may further comprise an appropriate conversion to the appropriate dosage form (and/or appropriate packaging of the dosage form). For example U.S. Pat. No. 7,323,493 may describe processed to an appropriate tablet form (see Examples 1 to 8), which may be a gelatin capsule.

1. A process for the preparation of a compound of formula III, 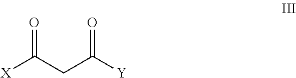
or a derivative thereof, wherein:
X represents hydrogen or C₁₋₆ alkyl optionally substituted by one or more halo atoms;
Y represents aryl or heteroaryl substituted by at least one —OH group;
which process comprises reaction of a compound of formula VII,
Y—C(O)—CH₃ VII
or a derivative thereof, wherein Y is as defined above, characterised in that the requisite —OH substituent thereon is not protected, with a compound of formula VIII,
X—B¹ VIII
or a derivative thereof, wherein:
X is as defined above;
B¹ represents —C≡N or —C(O)L¹;
L¹ is a suitable leaving group, such as halo or —OC₁₋₆ alkyl,
in the presence of base, wherein the base comprises an alkali metal alkoxide, in which the alkyl moiety of the alkoxide is a branched C₃₋₆ alkyl group, or an equivalent base thereof.

2. A process for the preparation of a compound of formula III, as defined in claim 1, comprising a process as claimed in claim 1, followed by crystallisation or precipitation of the compound, in a solvent system.

3. A process for the isolation of a compound of formula III (as defined in claim 1), which comprises crystallisation or precipitation as defined in claim 2.

4. A process as claimed in claim 2 or claim 3, wherein the solvent system comprises a mixture of water and a weak organic acid.

5. A process as claimed in claim 4, wherein the weak organic acid is acetic acid.

6. A product obtainable by the process of any one of claims 2 to 5.

7. A compound of formula III, as defined in claim 1, wherein the average particle size is at least 250×150 μM.

8. A compound as claimed in claim 7, wherein the average particle size is at least 400×300 μM.

9. A process for the preparation of a compound of formula I, 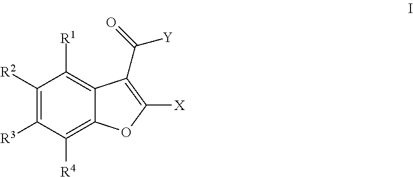
wherein R¹, R², R³ and R⁴ independently represent hydrogen, halo, —NO₂, —CN, —C(O)₂R^{x1}, —OR^{x2}, —SR^{x3}, —S(O)R^{x4}, —S(O)₂R^{x5}, —N(R^{x6})R^{x7}, —N(R^{x8})C(O)R^{x9}, —N(R^{x10})S(O)₂R^{x11} or R^{x12};
X represents hydrogen or C₁₋₆ alkyl optionally substituted by one or more halo (e.g. fluoro) atoms;
Y represents aryl or heteroaryl substituted by at least one (e.g. one) —OH group;
R^{x1}, R^{x2}, R^{x3}, R^{x6}, R^{x7}, R^{x8}, R^{x9} and R^{x10} independently represent hydrogen or C₁₋₆ alkyl optionally substituted by one or more halo (e.g. fluoro) atoms;
R^{x4}, R^{x5}, R^{x11} and R^{x12} independently represent C₁₋₆ alkyl optionally substituted by one or more halo (e.g. fluoro) atoms;
which process comprises reaction of a compound of formula II, 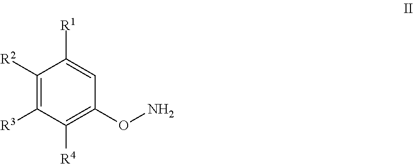
or a protected derivative or salt thereof, wherein R¹, R², R³, R⁴ are as defined above, with a compound of formula III, as claimed in any one of claims 6 to 8, or as prepared by a process as claimed in any one of claims 1 to 5.

10. A process for the preparation of a compound of formula I as defined in claim 9, characterised in that the reaction is performed as a “one-pot” procedure.

11. A process for the preparation of a compound of formula I as defined in claim 9, but characterised in that R² represents —NO₂, which process comprises reaction of a compound of formula II as defined in claim 9, or a protected derivative or salt thereof, but in which R² represents —NO₂.

12. A process for the preparation of a compound of formula I as defined in claim 9, characterised in that the process is performed in the absence of an acylating reagent.

13. A process for preparing Dronedarone, or a salt thereof, which process is characterised in that it includes as a process step a process as claimed in any one of claims 1 to 12.

14. A process for preparing a pharmaceutical formulation comprising Dronedarone, or a salt thereof, which process is characterised in that it includes as a process step a process as claimed in any one of claims 1 to 12.

15. A process for the preparation of Dronedarone, or a salt thereof, as claimed in claim 14, which comprises (in any order):
1) a process for the preparation of 2-butyl-3-(4-hydroxybenzoyl)-5-nitrobenzofuran as claimed in any one of claims 9 to 13;
2) conversion of the nitro (—NO₂) group to a methylsulfonylamino (—NHS(O)₂CH₃) group;
3) conversion of the —OH group to the —O—(CH₂)₃—N(C₄H₉)₂ group; and
4) if necessary/required, conversion of any free base of Dronedarone so formed to a salt.

16. A process as claimed in claim 15, wherein step (1) comprises the preparation of 2-butyl-3-(4-hydroxybenzoyl)-5-nitrobenzofuran, which is followed by step (3), then step (2), then step (4).

17. A process for the preparation of a pharmaceutical formulation comprising Dronedarone, or a salt thereof, which process comprises a process for the preparation of Dronedarone, or, a salt thereof, as claimed in claim 13, 14, 15 or 16, followed by bringing into association Dronedarone (or a salt thereof) so formed, with (a) pharmaceutically-acceptable excipient(s), adjuvant(s), diluent(s) or carrier(s).

18. A process for the preparation of a pharmaceutical formulation comprising Dronedarone, or a salt thereof, which process comprises a process for the preparation of Dronedarone, or, a salt thereof, as claimed in claim 13, 14 or 16, followed by bringing into association Dronedarone (or a salt thereof), with a pharmaceutically acceptable non-ionic hydrophilic surfactant selected from poloxamers, and, optionally, one or more pharmaceutical excipients.

19. A process for the preparation of an intermediate of Dronedarone, or a salt thereof, which process comprises a process step as claimed in any one of claims 9 to 13, followed by any one or more process steps disclosed in (1), (2) and (3) described in claim 15.

20. A process or compound substantially as described herein with reference to the examples.
